(19) United States
(12) Patent Application Publication
(10) Pub. No.: US 2025/0100988 A1
(43) Pub. Date: Mar. 27, 2025

(54) PREPARATION OF A COMPOUND FOR THE TREATMENT OF GOUT OR HYPERURICEMIA

(71) Applicant: Arthrosi Therapeutics, Inc., San Diego, CA (US)

(72) Inventors: Shunqi YAN, San Diego, CA (US); Litain YEH, San Diego, CA (US); Johny Sau-Hoi NG, San Diego, CA (US); Shuai PAN, Shanghai (CN); Minghua GAN, Shanghai (CN); Yueming YE, Shanghai (CN)

(21) Appl. No.: 18/725,070

(22) PCT Filed: Dec. 28, 2022
(86) PCT No.: PCT/CN2022/142846

§ 371 Date: Jun. 27, 2024

(30) Foreign Application Priority Data
Dec. 30, 2021 (WO) PCT/CN2021/143104

(51) Int. Cl. C07D 307/80; B01J 31/22; B01J 31/24; C07C 39/04; C07C 43/23; C07C 49/84; C07C 69/78; C07C 215/76

(52) U.S. Cl. CPC C07D 307/80; B01J 31/2295; B01J 31/2409; C07C 39/04; C07C 43/23; C07C 49/84; C07C 69/78; C07C 215/76; B01J 2231/324; B01J 2531/004; B01J 2531/824; B01J 2531/842; C07C 2601/16

(57) ABSTRACT

Described herein is the preparation of a compound for treating gout or hyperuricemia and chemical intermediates used in the synthetic process.

### CROSS-REFERENCE

This application claims benefit of PCT/CN2021/143104, filed on Dec. 30, 2021, which is herein incorporated by reference in its entirety.

### BACKGROUND OF THE INVENTION

Hyperuricemia is caused by the overproduction or under-excretion of uric acid, and is considered to be a causative factor of several diseases that significantly impair the quality of life. For example, hyperuricemia is considered the causative factor of gout—the most prevalent form of inflammatory arthritis, characterized by severe pain and tenderness in joints caused by urate crystal accumulation. The identification of a gout/hyperuricemia drug effective in lowering serum uric acid (sUA) with reduced toxicity represents an unmet medical need that would have beneficial impact on patients.

### SUMMARY OF THE INVENTION

Described herein are processes for the synthesis of a compound for the treatment of gout or hyperuricemia, wherein the compound is (3,5-dibromo-4-hydroxyphenyl)(2-(1-hydroxyethyl)benzofuran-3-yl-4,5,6,7-d₄)methanone (Compound 1), or a pharmaceutically acceptable salt thereof.

In one aspect is a process for the preparation of (3,5-dibromo-4-hydroxyphenyl)(2-(1-hydroxyethyl)benzofuran-3-yl-4,5,6,7-d₄)methanone (Compound 1):

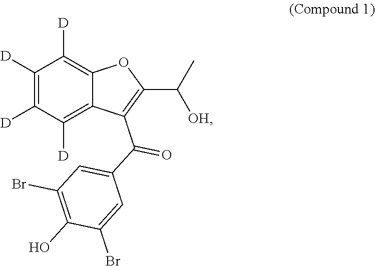 [SMILES: [2H]c1c([2H])c([2H])c2c(C(=O)c3cc(Br)c(O)c(Br)c3)c(C(C)O)oc2c1[2H]]

comprising contacting a compound with the structure:

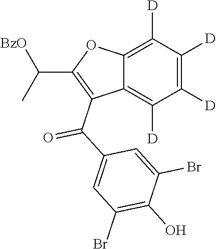

with cesium carbonate in the presence of a solvent. In some embodiments, the solvent is selected from methanol, ethanol, isopropanol, butanol, water, acetone, acetonitrile, dimethylformamide, dimethyl sulfoxide, N-methyl-2-pyrrolidone, acetic acid, and combinations thereof. In some embodiments, the solvent is selected from methanol.

In some embodiments of a process for the preparation of (3,5-dibromo-4-hydroxyphenyl)(2-(1-hydroxyethyl)benzofuran-3-yl-4,5,6,7-d₄)methanone (Compound 1), the compound with the structure:

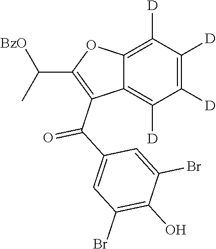

is prepared by a process comprising contacting a compound with the structure:

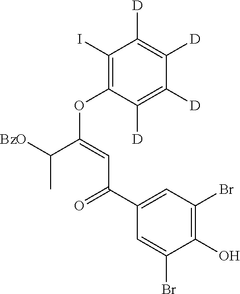

with a base and palladium catalyst in the presence of a solvent. In some embodiments, the base is selected from potassium carbonate, silver carbonate, sodium carbonate, cesium carbonate, sodium bicarbonate, triethylamine, diisopropylethylamine, 1,8-diazabicyclo(5.4.0)undec-7-ene, and 1,4-diazabicyclo[2.2.2]octane. In some embodiments, the base is potassium carbonate. In some embodiments, the palladium catalyst is selected from Pd(dppf)Cl₂, PdCl₂, Pd(OAc)₂, Pd(Ph₃P)₄, Pd₂(dba)₃, and Pd/C. In some embodiments, the palladium catalyst is Pd(dppf)Cl₂. In some embodiments, the ligand is selected from Ph₃P, BINAP, DPEphos, S-Phos, Xantphos, dtbpf, and Mephos. In some embodiments, the solvent is selected from dimethyl sulfoxide, dimethylformamide, N-methyl-2-pyrrolidone, acetone, acetonitrile, sulfolane, tetrahydrofuran, and toluene. In some embodiments, the solvent is dimethyl sulfoxide.

In some embodiments of a process for the preparation of (3,5-dibromo-4-hydroxyphenyl)(2-(1-hydroxyethyl)benzofuran-3-yl-4,5,6,7-d₄)methanone (Compound 1), the compound with the structure:

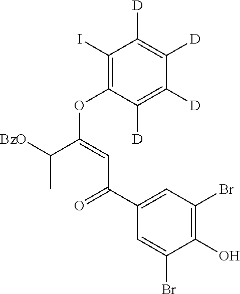

is prepared by a process comprising contacting a compound with the structure:

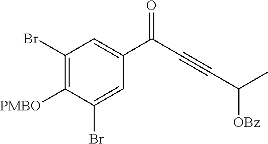

with a compound with the structure:

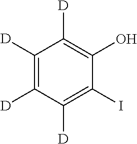 [SMILES: [2H]c1c([2H])c([2H])c(I)c(O)c1[2H]]

and potassium phosphate in the presence of a solvent followed by contact with trifluoroacetic acid. In some embodiments, the solvent is selected from dichloromethane, chloroform, acetonitrile, toluene, ethyl acetate, and tetrahydrofuran. In some embodiments, the solvent is dichloromethane.

In some embodiments of a process for the preparation of (3,5-dibromo-4-hydroxyphenyl)(2-(1-hydroxyethyl)benzofuran-3-yl-4,5,6,7-d₄)methanone (Compound 1), the compound with the structure:

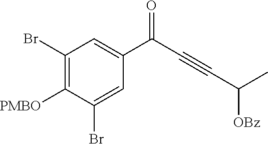

is prepared by a process comprising contacting a compound with the structure:

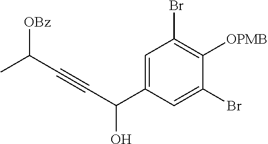

with sodium bicarbonate, potassium bromide, (2,2,6,6-tetramethylpiperidin-1-yl)oxyl (TEMPO), and sodium hypochlorite.

In some embodiments of a process for the preparation of (3,5-dibromo-4-hydroxyphenyl)(2-(1-hydroxyethyl)benzofuran-3-yl-4,5,6,7-d₄)methanone (Compound 1), the compound with the structure:

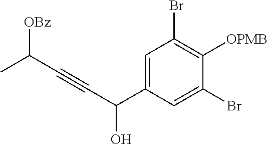

is prepared by a process comprising contacting a compound with the structure:

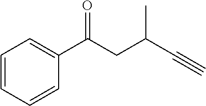

with a compound with the structure:

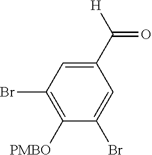

and a base in the presence of a solvent. In some embodiments, the base is selected from lithium diisopropylamide, LiHMDS, NaHMDS, KHMDS, t-BuOK, t-BuONa, t-BuOLi, and NaH. In some embodiments, the base is lithium diisopropylamide. In some embodiments, the solvent is selected from tetrahydrofuran, 2-methyltetrahydrofuran, dioxane, and toluene. In some embodiments, the solvent is tetrahydrofuran.

In some embodiments of a process for the preparation of (3,5-dibromo-4-hydroxyphenyl)(2-(1-hydroxyethyl)benzofuran-3-yl-4,5,6,7-d₄)methanone (Compound 1), the compound with the structure:

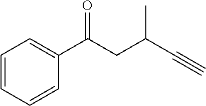

is prepared by a process comprising contacting a compound with the structure:

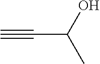

with benzoyl chloride and a base in the presence of a solvent. In some embodiments, the base is selected from pyridine, potassium carbonate, sodium hydroxide, triethylamine, diisopropylethylamine, 1,8-diazabicyclo(5.4.0)undec-7-ene, 1,4-diazabicyclo[2.2.2]octane, and lutidine. In some embodiments, the base is pyridine. In some embodiments, the solvent is selected from dichloromethane, tetrahydrofuran, 2-methyltetrahydrofuran, acetonitrile, dimethylformamide, dimethyl sulfoxide, and N-methyl-2-pyrrolidone. In some embodiments, the solvent is dichloromethane.

In some embodiments of a process for the preparation of (3,5-dibromo-4-hydroxyphenyl)(2-(1-hydroxyethyl)benzofuran-3-yl-4,5,6,7-d₄)methanone (Compound 1), the compound with the structure:

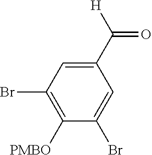

is prepared by a process comprising contacting a compound with the structure:

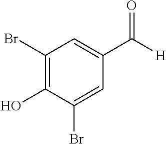 [SMILES: O=Cc1cc(Br)c(O)c(Br)c1]

with 4-methoxybenzyl chloride and a base in the presence of a solvent. In some embodiments, the base is selected from potassium carbonate, sodium hydride, sodium carbonate, sodium hydroxide, potassium hydroxide, lithium hydroxide, triethylamine, diisopropylethylamine, and pyridine. In some embodiments, the base is potassium carbonate. In some embodiments, the solvent is selected from acetonitrile, dichloromethane, chloroform, tetrahydrofuran, 2-methyltetrahydrofuran, dimethylformamide, dimethyl sulfoxide, and N-methyl-2-pyrrolidone. In some embodiments, the solvent is acetonitrile.

In some embodiments of a process for the preparation of (3,5-dibromo-4-hydroxyphenyl)(2-(1-hydroxyethyl)benzofuran-3-yl-4,5,6,7-d₄)methanone (Compound 1), the compound with the structure:

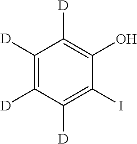 [SMILES: [2H]c1c([2H])c([2H])c(I)c(O)c1[2H]]

is prepared by a process comprising contacting a compound with the structure:

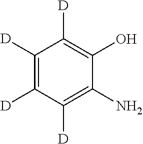 [SMILES: [2H]c1c([2H])c([2H])c(O)c(N)c1[2H]]

with sodium nitrite, potassium iodide, and toluenesulfonic acid.

In some embodiments of a process for the preparation of (3,5-dibromo-4-hydroxyphenyl)(2-(1-hydroxyethyl)benzofuran-3-yl-4,5,6,7-d₄)methanone (Compound 1), the compound with the structure:

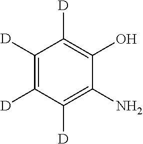 [SMILES: [2H]c1c([2H])c([2H])c(O)c(N)c1[2H]]

is prepared by a process comprising contacting a compound with the structure:

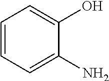

with 10% platinum on carbon and deuterium oxide.

Further disclosed herein, is a process for the preparation of (3,5-dibromo-4-hydroxyphenyl)(2-(1-hydroxyethyl)benzofuran-3-yl-4,5,6,7-d₄)methanone (Compound 1), comprising:
A) the reaction of a compound with the structure:

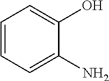

with 10% platinum on carbon and deuterium oxide to produce a compound with the structure:

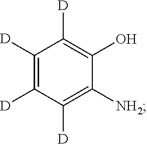 [SMILES: [2H]c1c([2H])c([2H])c(O)c(N)c1[2H]]

B) followed by the reaction of the compound with the structure:

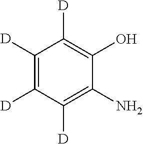 [SMILES: [2H]c1c([2H])c([2H])c(O)c(N)c1[2H]]

with sodium nitrite, potassium iodide, and toluenesulfonic acid to produce a compound with the structure:

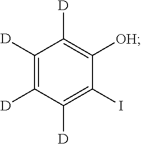 [SMILES: [2H]c1c([2H])c([2H])c(I)c(O)c1[2H]]

C) the reaction of the compound with the structure:

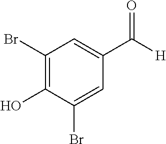 [SMILES: O=Cc1cc(Br)c(O)c(Br)c1]

with 4-methoxybenzyl chloride and potassium carbonate to produce a compound with the structure:

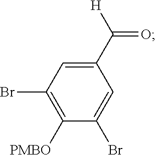

D) the reaction of the compound with the structure:

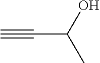

with benzoyl chloride and pyridine to produce a compound with the structure:

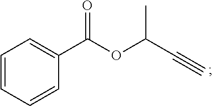

E) followed by the reaction of the compounds with the structure:

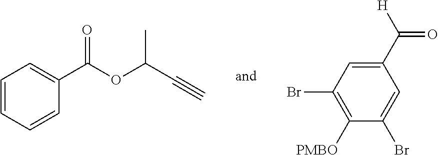

with lithium diisopropylamide to produce a compound with the structure:

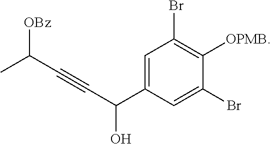

F) followed by the reaction of the compound with the structure:

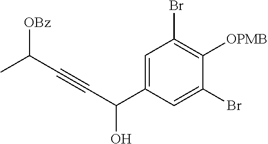

with sodium bicarbonate, potassium bromide, (2,2,6,6-tetramethylpiperidin-1-yl)oxyl (TEMPO), and sodium hypochlorite to produce a compound with the structure:

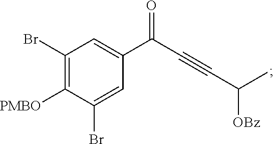

G) followed by the reaction of the compounds with the structures:

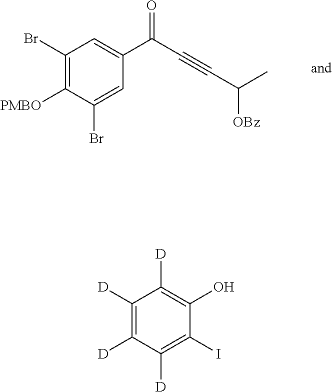

and potassium phosphate followed by contact with trifluoroacetic acid to produce a compound with the structure:

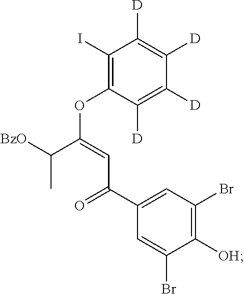

H) followed by the reaction of the compound with the structure:

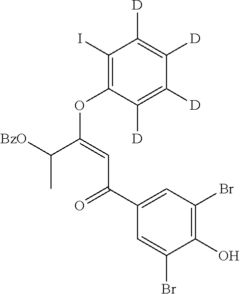

with potassium carbonate and Pd(dppf)Cl₂ to produce a compound with the structure:

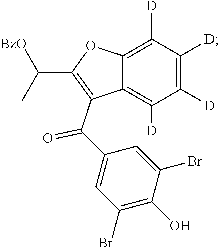

I) followed by the reaction of the compound with the structure:

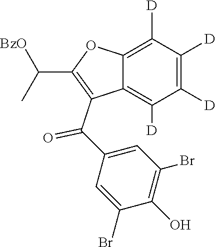

with cesium carbonate to produce a compound with the structure:

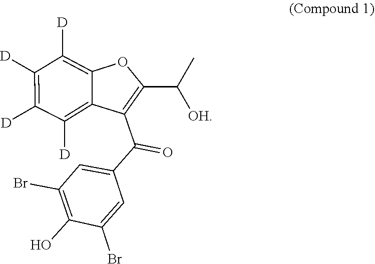 [SMILES: [2H]c1c([2H])c([2H])c2c(C(=O)c3cc(Br)c(O)c(Br)c3)c(C(C)O)oc2c1[2H]]

Further disclosed herein is a compound having a structure selected from:

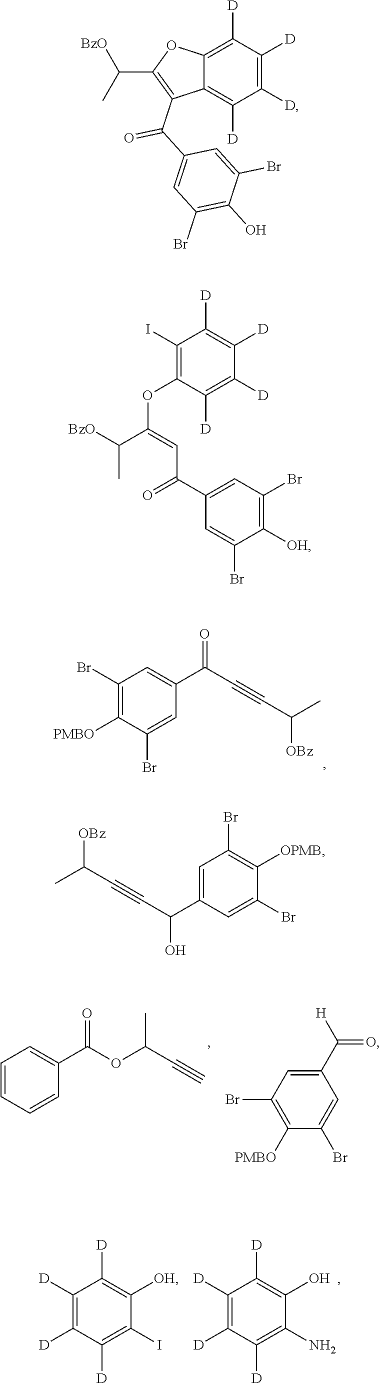

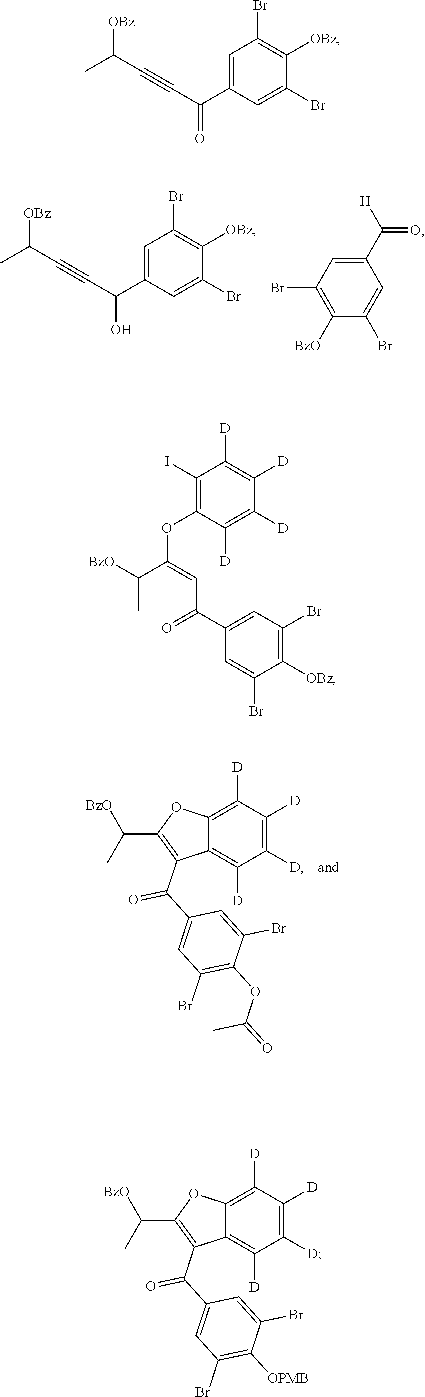

or a pharmaceutically acceptable salt thereof.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION OF THE INVENTION

Good manufacturing practices are usually required for large scale manufacture of clinically useful drug candidates. Provided herein are certain processes and methods for the manufacture of (3,5-dibromo-4-hydroxyphenyl)(2-(1-hydroxyethyl)benzofuran-3-yl-4,5,6,7-d₄)methanone (Compound 1), or a pharmaceutically acceptable salt or co-crystal thereof.

### Definitions

As used in the specification and appended claims, unless specified to the contrary, the following terms have the meaning indicated below.

As used herein and in the appended claims, the singular forms “a,” “and,” and “the” include plural referents unless the context clearly dictates otherwise. Thus, for example, reference to “an agent” includes a plurality of such agents, and reference to “the cell” includes reference to one or more cells (or to a plurality of cells) and equivalents thereof.

When ranges are used herein for physical properties, such as molecular weight, or chemical properties, such as chemical formulae, all combinations and subcombinations of ranges and specific embodiments therein are intended to be included.

The term “about” when referring to a number or a numerical range means that the number or numerical range referred to is an approximation within experimental variability (or within statistical experimental error), and thus the number or numerical range varies between 1% and 15% of the stated number or numerical range.

The term “comprising” (and related terms such as “comprise” or “comprises” or “having” or “including”) is not intended to exclude that which in other certain embodiments, for example, an embodiment of any composition of matter, composition, method, or process, or the like, described herein, “consist of” or “consist essentially of” the described features.

The term “subject” or “patient” encompasses mammals and non-mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. In one embodiment of the methods and compositions provided herein, the mammal is a human.

As used herein, “treatment” or “treating” or “palliating” or “ameliorating” are used interchangeably herein. These terms refers to an approach for obtaining beneficial or desired results including but not limited to therapeutic benefit and/or a prophylactic benefit. By “therapeutic benefit” is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient is still afflicted with the underlying disorder. For prophylactic benefit, the compositions are administered to a patient at risk of developing a particular disease, or to a patient reporting one or more of the physiological symptoms of a disease, even though a diagnosis of this disease has been made.

“Pharmaceutically acceptable salt” includes both acid and base addition salts. A pharmaceutically acceptable salt of any one of the compounds described herein is intended to encompass any and all pharmaceutically suitable salt forms. Preferred pharmaceutically acceptable salts of the compounds described herein are pharmaceutically acceptable acid addition salts and pharmaceutically acceptable base addition salts.

“Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid, hydroiodic acid, hydrofluoric acid, phosphorous acid, and the like. Also included are salts that are formed with organic acids such as aliphatic mono- and dicarboxylic acids, phenyl-substituted alkanoic acids, hydroxy alkanoic acids, alkanedioic acids, aromatic acids, aliphatic and. aromatic sulfonic acids, etc. and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Exemplary salts thus include sulfates, pyrosulfates, bisulfates, sulfites, bisulfites, nitrates, phosphates, monohydrogenphosphates, dihydrogenphosphates, metaphosphates, pyrophosphates, chlorides, bromides, iodides, acetates, trifluoroacetates, propionates, caprylates, isobutyrates, oxalates, malonates, succinate suberates, sebacates, fumarates, maleates, mandelates, benzoates, chlorobenzoates, methylbenzoates, dinitrobenzoates, phthalates, benzenesulfonates, toluenesulfonates, phenylacetates, citrates, lactates, malates, tartrates, methanesulfonates, and the like. Also contemplated are salts of amino acids, such as arginates, gluconates, and galacturonates (see, for example, Berge S. M. et al., “Pharmaceutical Salts,” Journal of Pharmaceutical Science, 66:1-19 (1997)). Acid addition salts of basic compounds are prepared by contacting the free base forms with a sufficient amount of the desired acid to produce the salt.

“Pharmaceutically acceptable base addition salt” refers to those salts that retain the biological effectiveness and properties of the free acids, which are not biologically or otherwise undesirable. These salts are prepared from addition of an inorganic base or an organic base to the free acid. In some embodiments, pharmaceutically acceptable base addition salts are formed with metals or amines, such as alkali and alkaline earth metals or organic amines. Salts derived from inorganic bases include, but are not limited to, sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, for example, isopropylamine, trimethylamine, diethylamine, triethylamine, tripropylamine, ethanolamine, diethanolamine, 2-dimethylaminoethanol, 2-diethylaminoethanol, dicyclohexylamine, lysine, arginine, histidine, caffeine, procaine, N,N-dibenzylethylenediamine, chloroprocaine, hydrabamine, choline, betaine, ethylenediamine, ethylenedianiline, N-methylglucamine, glucosamine, methylglucamine, theobromine, purines, piperazine, piperidine, N-ethylpiperidine, polyamine resins and the like. See Berge et al., supra.

The term “pharmaceutical combination” as used herein, means a product that results from the mixing or combining of more than one active ingredient and includes both fixed and non-fixed combinations of the active ingredients. The term “fixed combination” means that the active ingredients, are both administered to a patient simultaneously in the form of a single entity or dosage. The term “non-fixed combination” means that the active ingredients, are administered to a patient as separate entities either simultaneously, concurrently or sequentially with no specific intervening time limits, wherein such administration provides effective levels of the two compounds in the body of the patient. The latter also applies to cocktail therapy, e.g. the administration of three or more active ingredients.

The terms “co-administration” or the like, as used herein, are meant to encompass administration of the selected therapeutic agents to a single patient, and are intended to include treatment regimens in which the agents are administered by the same or different route of administration or at the same or different time.

The term “activator” is used in this specification to denote any molecular species that results in activation of the indicated receptor, regardless of whether the species itself binds to the receptor or a metabolite of the species binds to the receptor when the species is administered topically. Thus, the activator can be a ligand of the receptor or it can be an activator that is metabolized to the ligand of the receptor, i.e., a metabolite that is formed in tissue and is the actual ligand.

The term “antagonist” as used herein, refers to a small-molecule agent that binds to a nuclear hormone receptor and subsequently decreases the agonist induced transcriptional activity of the nuclear hormone receptor.

The term “agonist” as used herein, refers to a small-molecule agent that binds to a nuclear hormone receptor and subsequently increases nuclear hormone receptor transcriptional activity in the absence of a known agonist.

The term “inverse agonist” as used herein, refers to a small-molecule agent that binds to a nuclear hormone receptor and subsequently decreases the basal level of nuclear hormone receptor transcriptional activity that is present in the absence of a known agonist.

The term “modulate,” as used herein, means to interact with a target protein either directly or indirectly so as to alter the activity of the target protein, including, by way of example only, to inhibit the activity of the target, or to limit or reduce the activity of the target.

As used herein, the term “modulator” refers to a compound that alters an activity of a target. For example, a modulator can cause an increase or decrease in the magnitude of a certain activity of a target compared to the magnitude of the activity in the absence of the modulator. In certain embodiments, a modulator is an inhibitor, which decreases the magnitude of one or more activities of a target. In certain embodiments, an inhibitor completely prevents one or more activities of a target.

### Compounds

In some embodiments, the compound for the treatment of gout or hyperuricemia described herein is (3,5-dibromo-4-hydroxyphenyl)(2-(1-hydroxyethyl)benzofuran-3-yl-4,5,6,7-d₄)methanone (Compound 1), or a pharmaceutically acceptable salt or co-crystal thereof. Compound 1 has the structure:

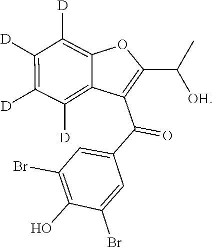 [SMILES: [2H]c1c([2H])c([2H])c2c(C(=O)c3cc(Br)c(O)c(Br)c3)c(C(C)O)oc2c1[2H]]

In some embodiments, the starting material for the synthesis of Compound 1 is

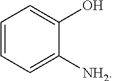

In some embodiments, an intermediate in the synthesis of Compound 1 is

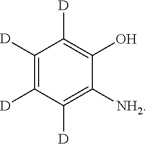 [SMILES: [2H]c1c([2H])c([2H])c(O)c(N)c1[2H]]

In some embodiments, an intermediate in the synthesis of Compound 1 is

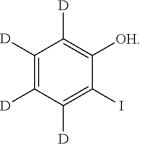 [SMILES: [2H]c1c([2H])c([2H])c(I)c(O)c1[2H]]

In some embodiments, a starting material in the synthesis of Compound 1 is

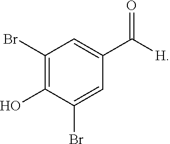

In some embodiments, an intermediate in the synthesis of Compound 1 is

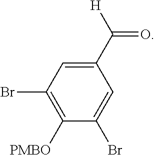

In some embodiments, a starting material in the synthesis of Compound 1 is

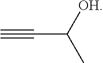

In some embodiments, an intermediate in the synthesis of Compound 1 is

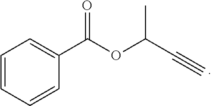

In some embodiments, an intermediate in the synthesis of Compound 1 is

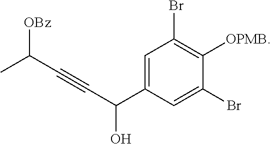

In some embodiments, an intermediate in the synthesis of Compound 1 is

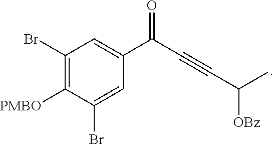

In some embodiments, an intermediate in the synthesis of Compound 1 is

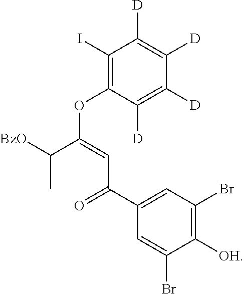

In some embodiments, an intermediate in the synthesis of Compound 1 is

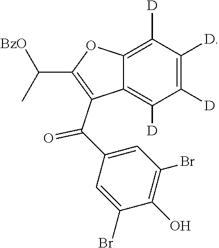

In some embodiments, an intermediate in the synthesis of Compound 1 is

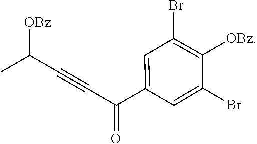

In some embodiments, an intermediate in the synthesis of Compound 1 is

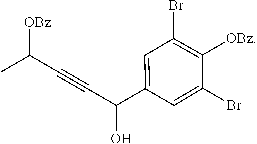

In some embodiments, an intermediate in the synthesis of Compound 1 is

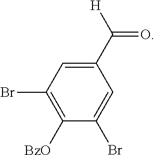

In some embodiments, an intermediate in the synthesis of Compound 1 is

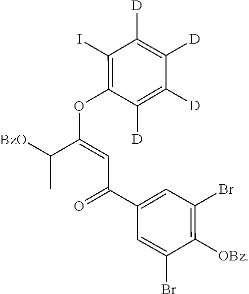

In some embodiments, an intermediate in the synthesis of Compound 1 is

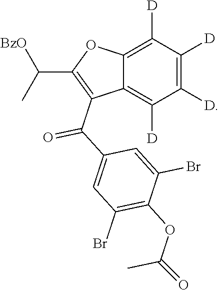

In some embodiments, an intermediate in the synthesis of Compound 1 is

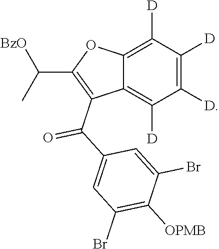

### Further Forms of Compounds

The compounds described herein may in some cases exist as diastereomers, enantiomers, or other stereoisomeric forms. The compounds presented herein include all diastereomeric, enantiomeric, and epimeric forms as well as the appropriate mixtures thereof. Separation of stereoisomers may be performed by chromatography or by the forming diastereomeric and separation by recrystallization, or chromatography, or any combination thereof (Jean Jacques, Andre Collet, Samuel H. Wilen, “Enantiomers, Racemates and Resolutions”, John Wiley And Sons, Inc., 1981, herein incorporated by reference for this disclosure). Stereoisomers may also be obtained by stereoselective synthesis.

In some situations, compounds may exist as tautomers. All tautomers are included within the formulas described herein.

### Pharmaceutically Acceptable Salts

In some embodiments, the compounds described herein exist as their pharmaceutically acceptable salts. In some embodiments, the methods disclosed herein include methods of treating diseases by administering such pharmaceutically acceptable salts. In some embodiments, the methods disclosed herein include methods of treating diseases by administering such pharmaceutically acceptable salts as pharmaceutical compositions.

In some embodiments, the compounds described herein possess acidic or basic groups and therefore react with any of a number of inorganic or organic bases, and inorganic and organic acids, to form a pharmaceutically acceptable salt. In some embodiments, these salts are prepared in situ during the final isolation and purification of the compounds of the invention, or by separately reacting a purified compound in its free form with a suitable acid or base, and isolating the salt thus formed.

In some embodiments, the pharmaceutically acceptable salt of Compound 1 is an acetate, benzoate, besylate, bitartrate, carbonate, citrate, fumarate, gluconate, hydrobromide, hydrochloride, maleate, mesylate, nitrate, phosphate, salicylate, succinate, sulfate, or tartrate salt. In some embodiments, the pharmaceutically acceptable salt of Compound 1 is a mono-hydrochloride salt. In further embodiments, the pharmaceutically acceptable salt of Compound 1 is a mono-hydrochloride salt.

### Solvates

In some embodiments, the compounds described herein exist as solvates. The invention provides for methods of treating diseases by administering such solvates. The invention further provides for methods of treating diseases by administering such solvates as pharmaceutical compositions.

Solvates contain either stoichiometric or non-stoichiometric amounts of a solvent, and, in some embodiments, are formed during the process of crystallization with pharmaceutically acceptable solvents such as water, ethanol, and the like. Hydrates are formed when the solvent is water, or alcoholates are formed when the solvent is alcohol. Solvates of the compounds described herein are conveniently prepared or formed during the processes described herein. By way of example only, hydrates of the compounds described herein are conveniently prepared by recrystallization from an aqueous/organic solvent mixture, using organic solvents including, but not limited to, dioxane, tetrahydrofuran or methanol. In addition, the compounds provided herein exist in unsolvated as well as solvated forms. In general, the solvated forms are considered equivalent to the unsolvated forms for the purposes of the compounds and methods provided herein.

### Labeled Compounds

In some embodiments, the compounds described herein exist in their isotopically-labeled forms. In some embodiments, the methods disclosed herein include methods of treating diseases by administering such isotopically-labeled compounds. In some embodiments, the methods disclosed herein include methods of treating diseases by administering such isotopically-labeled compounds as pharmaceutical compositions. Thus, in some embodiments, the compounds disclosed herein include isotopically-labeled compounds, which are identical to those recited herein, but for the fact that one or more atoms are replaced by an atom having an atomic mass or mass number different from the atomic mass or mass number usually found in nature. Examples of isotopes that are incorporated into compounds of the invention include isotopes of hydrogen, carbon, nitrogen, oxygen, phosphorous, sulfur, fluorine and chloride, such as ²H, ³H, ¹³C, ¹⁴C, ¹⁵N, ¹⁸O, ¹⁷O, ³¹P, ³²P, ³⁵S, ¹⁸F, and ³⁶Cl, respectively. Compounds described herein, and pharmaceutically acceptable salts, esters, solvate, hydrates or derivatives thereof which contain the aforementioned isotopes and/or other isotopes of other atoms are within the scope of this invention. Certain isotopically-labeled compounds, for example those into which radioactive isotopes such as ³H and ¹⁴C are incorporated, are useful in drug and/or substrate tissue distribution assays. Tritiated, i. e., ³H and carbon-14, i. e., ¹⁴C, isotopes are particularly preferred for their ease of preparation and detectability. Further, substitution with heavy isotopes such as deuterium, i.e., ²H, produces certain therapeutic advantages resulting from greater metabolic stability, for example increased in vivo half-life or reduced dosage requirements. Increased levels of deuterium incorporation produce a detectable kinetic isotope effect (KIE) that may affect the pharmacokinetic, pharmacologic and/or toxicologic parameters of Compound 1 in comparison to Compound 1 having naturally occurring levels of deuterium. In some embodiments, the isotopically labeled compound, or a pharmaceutically acceptable salt thereof, is prepared by any suitable method.

In some embodiments, at least one hydrogen in Compound 1 is replaced with deuterium.

In some embodiments, the compounds described herein are labeled by other means, including, but not limited to, the use of chromophores or fluorescent moieties, bioluminescent labels, or chemiluminescent labels.

### Process for Preparation

In some embodiments, the synthesis of compounds described herein are accomplished using means described in the chemical literature, using the methods described herein, or by a combination thereof. In addition, solvents, temperatures and other reaction conditions presented herein may vary.

In other embodiments, the starting materials and reagents used for the synthesis of the compounds described herein are synthesized or are obtained from commercial sources, such as, but not limited to, Sigma-Aldrich, FischerScientific (Fischer Chemicals), and AcrosOrganics. In further embodiments, the compounds described herein, and other related compounds having different substituents are synthesized using techniques and materials described herein as well as those that are recognized in the field, such as described, for example, in Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), Larock's Comprehensive Organic Transformations (VCH Publishers Inc., 1989), March, Advanced Organic Chemistry 4^{th} Ed., (Wiley 1992); Carey and Sundberg, Advanced Organic Chemistry 4^{th} Ed., Vols. A and B (Plenum 2000, 2001), and Green and Wuts, Protective Groups in Organic Synthesis 3^{rd} Ed., (Wiley 1999) (all of which are incorporated by reference for such disclosure). General methods for the preparation of compounds as disclosed herein may be derived from reactions and the reactions may be modified by the use of appropriate reagents and conditions, for the introduction of the various moieties found in the formulae as provided herein.

In some embodiments is a process for the preparation of (3,5-dibromo-4-hydroxyphenyl)(2-(1-hydroxyethyl)benzofuran-3-yl-4,5,6,7-d₄)methanone (Compound 1):

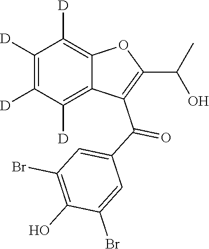 [SMILES: [2H]c1c([2H])c([2H])c2c(C(=O)c3cc(Br)c(O)c(Br)c3)c(C(C)O)oc2c1[2H]]

comprising contacting a compound with the structure:

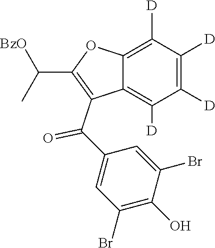

with cesium carbonate in the presence of a solvent. In some embodiments, the solvent is selected from methanol, ethanol, isopropanol, butanol, water, acetone, acetonitrile, dimethylformamide, dimethyl sulfoxide, N-methyl-2-pyrrolidone, acetic acid, and combinations thereof. In some embodiments, the solvent is methanol. In some embodiments, the solvent is ethanol. In some embodiments, the solvent is isopropanol. In some embodiments, the solvent is butanol. In some embodiments, the solvent is water. In some embodiments, the solvent is acetone. In some embodiments, the solvent is acetonitrile. In some embodiments, the solvent is dimethylformamide. In some embodiments, the solvent is dimethyl sulfoxide. In some embodiments, the solvent is N-methyl-2-pyrrolidone. In some embodiments, the solvent is acetic acid.

In some embodiments of a process for the preparation of (3,5-dibromo-4-hydroxyphenyl)(2-(1-hydroxyethyl)benzofuran-3-yl-4,5,6,7-d₄)methanone (Compound 1), the compound with the structure:

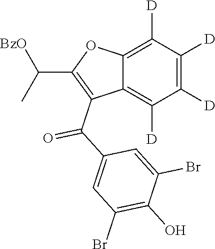

is prepared by a process comprising contacting a compound with the structure:

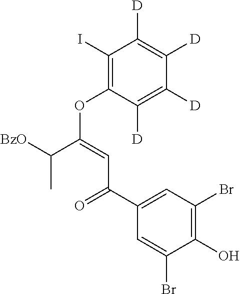

with a base and palladium catalyst in the presence of a solvent. In some embodiments, the base is selected from potassium carbonate, silver carbonate, sodium carbonate, cesium carbonate, sodium bicarbonate, triethylamine, diisopropylethylamine, 1,8-diazabicyclo(5.4.0)undec-7-ene, and 1,4-diazabicyclo[2.2.2]octane. In some embodiments, the base is potassium carbonate. In some embodiments, the base is silver carbonate. In some embodiments, the base is sodium carbonate. In some embodiments, the base is cesium carbonate. In some embodiments, the base is sodium bicarbonate. In some embodiments, the base is triethylamine. In some embodiments, the base is diisopropylethylamine. In some embodiments, the base is 1,8-diazabicyclo(5.4.0)undec-7-ene. In some embodiments, the base is 1,4-diazabicyclo[2.2.2]octane. In some embodiments, the palladium catalyst is selected from Pd(dppf)Cl₂, PdCl₂, Pd(OAc)₂, Pd(Ph₃P)₄, Pd₂(dba)₃, and Pd/C. In some embodiments, the palladium catalyst is Pd(dppf)Cl₂. In some embodiments, the palladium catalyst is PdCl₂. In some embodiments, the palladium catalyst is Pd(OAc)₂. In some embodiments, the palladium catalyst is Pd(Ph₃P)₄. In some embodiments, the palladium catalyst is Pd₂(dba)₃. In some embodiments, the palladium catalyst is Pd/C. In some embodiments, the ligand is selected from Ph₃P, BINAP, DPEphos, S-Phos, Xantphos, dtbpf, and Mephos. In some embodiments, the ligand is Ph₃P. In some embodiments, the ligand is BINAP. In some embodiments, the ligand is DPEphos. In some embodiments, the ligand is S-Phos. In some embodiments, the ligand is Xantphos. In some embodiments, the ligand is dtbpf. In some embodiments, the ligand is Mephos. In some embodiments, the solvent is selected from dimethyl sulfoxide, dimethylformamide, N-methyl-2-pyrrolidone, acetone, acetonitrile, sulfolane, tetrahydrofuran, and toluene. In some embodiments, the solvent is dimethyl sulfoxide. In some embodiments, the solvent is dimethylformamide. In some embodiments, the solvent is N-methyl-2-pyrrolidone. In some embodiments, the solvent is acetone. In some embodiments, the solvent is acetonitrile. In some embodiments, the solvent is sulfolane. In some embodiments, the solvent is tetrahydrofuran. In some embodiments, the solvent is toluene.

In some embodiments of a process for the preparation of (3,5-dibromo-4-hydroxyphenyl)(2-(1-hydroxyethyl)benzofuran-3-yl-4,5,6,7-d₄)methanone (Compound 1), the compound with the structure:

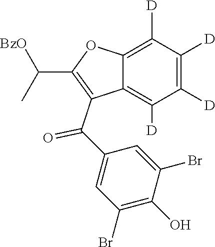

is prepared by a process comprising contacting a compound with the structure:

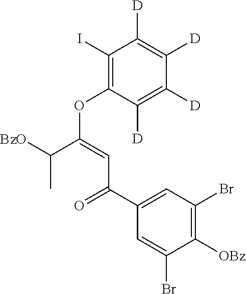

with a base and palladium catalyst in the presence of a solvent. In some embodiments, the base is selected from potassium carbonate, silver carbonate, sodium carbonate, cesium carbonate, sodium bicarbonate, triethylamine, diisopropylethylamine, 1,8-diazabicyclo(5.4.0)undec-7-ene, and 1,4-diazabicyclo[2.2.2]octane. In some embodiments, the base is triethylamine. In some embodiments, the base is potassium carbonate. In some embodiments, the base is silver carbonate. In some embodiments, the base is sodium carbonate. In some embodiments, the base is cesium carbonate. In some embodiments, the base is sodium bicarbonate. In some embodiments, the base is diisopropylethylamine. In some embodiments, the base is 1,8-diazabicyclo(5.4.0)undec-7-ene. In some embodiments, the base is 1,4-diazabicyclo[2.2.2]octane. In some embodiments, the palladium catalyst is selected from Pd(dppf)Cl₂, PdCl₂, Pd(OAc)₂, Pd(Ph₃P)₄, Pd₂(dba)₃, and Pd/C. In some embodiments, the palladium catalyst is Pd(dppf)Cl₂. In some embodiments, the palladium catalyst is PdCl₂. In some embodiments, the palladium catalyst is Pd(OAc)₂. In some embodiments, the palladium catalyst is Pd(Ph₃P)₄. In some embodiments, the palladium catalyst is Pd₂(dba)₃. In some embodiments, the palladium catalyst is Pd/C. In some embodiments, the ligand is selected from Ph₃P, BINAP, DPEphos, S-Phos, Xantphos, dtbpf, and Mephos. In some embodiments, the ligand is Ph₃P. In some embodiments, the ligand is BINAP. In some embodiments, the ligand is DPEphos. In some embodiments, the ligand is S-Phos. In some embodiments, the ligand is Xantphos. In some embodiments, the ligand is dtbpf. In some embodiments, the ligand is Mephos. In some embodiments, the solvent is selected from dimethyl sulfoxide, dimethylformamide, N-methyl-2-pyrrolidone, acetone, acetonitrile, sulfolane, tetrahydrofuran, and toluene. In some embodiments, the solvent is dimethyl sulfoxide. In some embodiments, the solvent is dimethylformamide. In some embodiments, the solvent is N-methyl-2-pyrrolidone. In some embodiments, the solvent is acetone. In some embodiments, the solvent is acetonitrile. In some embodiments, the solvent is sulfolane. In some embodiments, the solvent is tetrahydrofuran. In some embodiments, the solvent is toluene.

In some embodiments of a process for the preparation of (3,5-dibromo-4-hydroxyphenyl)(2-(1-hydroxyethyl)benzofuran-3-yl-4,5,6,7-d₄)methanone (Compound 1), the compound with the structure:

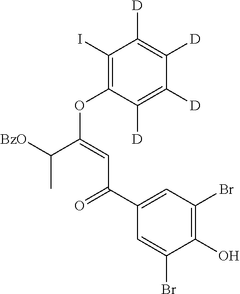

is prepared by a process comprising contacting a compound with the structure:

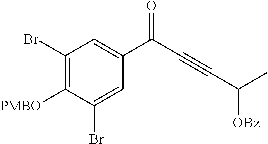

with a compound with the structure:

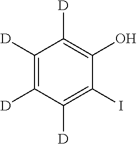 [SMILES: [2H]c1c([2H])c([2H])c(I)c(O)c1[2H]]

and potassium phosphate in the presence of a solvent followed by contact with trifluoroacetic acid. In some embodiments, the solvent is selected from dichloromethane, chloroform, acetonitrile, toluene, ethyl acetate, and tetrahydrofuran. In some embodiments, the solvent is dichloromethane. In some embodiments, the solvent is chloroform. In some embodiments, the solvent is acetonitrile. In some embodiments, the solvent is toluene. In some embodiments, the solvent is ethyl acetate. In some embodiments, the solvent is tetrahydrofuran.

In some embodiments of a process for the preparation of (3,5-dibromo-4-hydroxyphenyl)(2-(1-hydroxyethyl)benzofuran-3-yl-4,5,6,7-d₄)methanone (Compound 1), the compound with the structure:

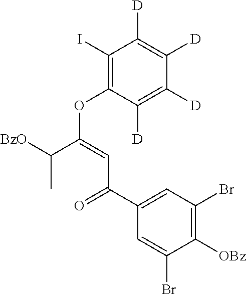

is prepared by a process comprising contacting a compound with the structure:

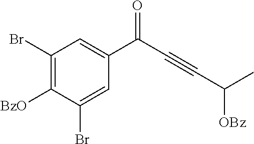

with a compound with the structure:

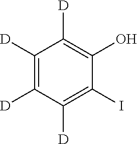 [SMILES: [2H]c1c([2H])c([2H])c(I)c(O)c1[2H]]

and potassium phosphate in the presence of a solvent. In some embodiments, the solvent is selected from dichloromethane, chloroform, acetonitrile, toluene, ethyl acetate, and tetrahydrofuran. In some embodiments, the solvent is dichloromethane. In some embodiments, the solvent is chloroform. In some embodiments, the solvent is acetonitrile. In some embodiments, the solvent is toluene. In some embodiments, the solvent is ethyl acetate. In some embodiments, the solvent is tetrahydrofuran.

In some embodiments of a process for the preparation of (3,5-dibromo-4-hydroxyphenyl)(2-(1-hydroxyethyl)benzofuran-3-yl-4,5,6,7-d₄)methanone (Compound 1), the compound with the structure:

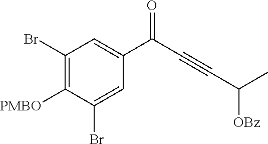

is prepared by a process comprising contacting a compound with the structure:

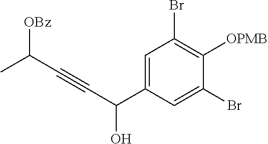

with sodium bicarbonate, potassium bromide, (2,2,6,6-tetramethylpiperidin-1-yl)oxyl (TEMPO), and sodium hypochlorite.

In some embodiments of a process for the preparation of (3,5-dibromo-4-hydroxyphenyl)(2-(1-hydroxyethyl)benzofuran-3-yl-4,5,6,7-d₄)methanone (Compound 1), the compound with the structure:

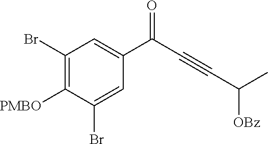

is prepared by a process comprising contacting a compound with the structure:

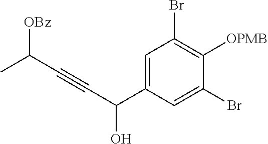

under Swern oxidation conditions.

In some embodiments of a process for the preparation of (3,5-dibromo-4-hydroxyphenyl)(2-(1-hydroxyethyl)benzofuran-3-yl-4,5,6,7-d₄)methanone (Compound 1), the compound with the structure:

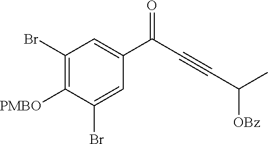

is prepared by a process comprising contacting a compound with the structure:

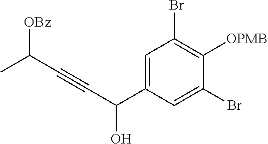

with MnO₂.

In some embodiments of a process for the preparation of (3,5-dibromo-4-hydroxyphenyl)(2-(1-hydroxyethyl)benzofuran-3-yl-4,5,6,7-d₄)methanone (Compound 1), the compound with the structure:

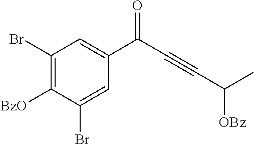

is prepared by a process comprising contacting a compound with the structure:

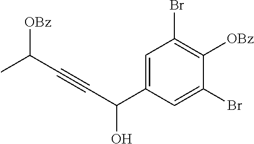

with sodium bicarbonate, potassium bromide, (2,2,6,6-tetramethylpiperidin-1-yl)oxyl (TEMPO), and sodium hypochlorite.

In some embodiments of a process for the preparation of (3,5-dibromo-4-hydroxyphenyl)(2-(1-hydroxyethyl)benzofuran-3-yl-4,5,6,7-d₄)methanone (Compound 1), the compound with the structure:

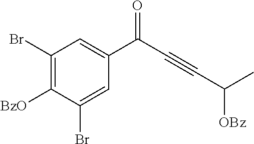

is prepared by a process comprising contacting a compound with the structure:

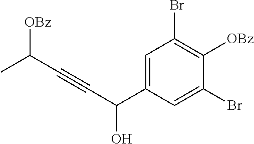

under Swern oxidation conditions.

In some embodiments of a process for the preparation of (3,5-dibromo-4-hydroxyphenyl)(2-(1-hydroxyethyl)benzofuran-3-yl-4,5,6,7-d₄)methanone (Compound 1), the compound with the structure:

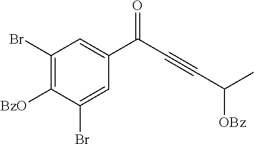

is prepared by a process comprising contacting a compound with the structure:

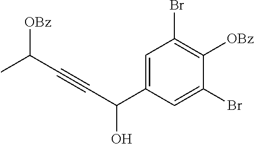

with MnO₂.

In some embodiments of a process for the preparation of (3,5-dibromo-4-hydroxyphenyl)(2-(1-hydroxyethyl)benzofuran-3-yl-4,5,6,7-d₄)methanone (Compound 1), the compound with the structure:

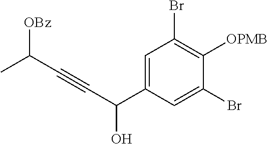

is prepared by a process comprising contacting a compound with the structure:

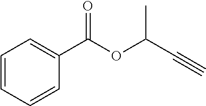

with a compound with the structure:

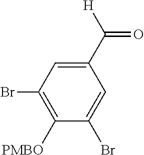

and a base in the presence of a solvent. In some embodiments, the base is selected from lithium diisopropylamide, LiHMDS, NaHMDS, KHMDS, t-BuOK, t-BuONa, t-BuOLi, and NaH. In some embodiments, the base is lithium diisopropylamide. In some embodiments, the base is LiHMDS. In some embodiments, the base is NaHMDS. In some embodiments, the base is KHMDS. In some embodiments, the base is t-BuOK. In some embodiments, the base is t-BuONa. In some embodiments, the base is t-BuOLi. In some embodiments, the base is NaH. In some embodiments, the solvent is selected from tetrahydrofuran, 2-methyltetrahydrofuran, dioxane, and toluene. In some embodiments, the solvent is tetrahydrofuran. In some embodiments, the solvent is 2-methyltetrahydrofuran. In some embodiments, the solvent is dioxane. In some embodiments, the solvent is toluene.

In some embodiments of a process for the preparation of (3,5-dibromo-4-hydroxyphenyl)(2-(1-hydroxyethyl)benzofuran-3-yl-4,5,6,7-d₄)methanone (Compound 1), the compound with the structure:

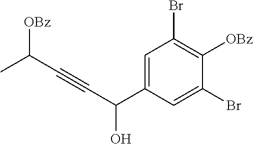

is prepared by a process comprising contacting a compound with the structure:

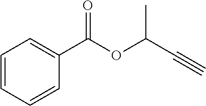

with a compound with the structure:

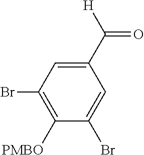

and a base in the presence of a solvent. In some embodiments, the base is selected from lithium diisopropylamide, LiHMDS, NaHMDS, KHMDS, t-BuOK, t-BuONa, t-BuOLi, and NaH. In some embodiments, the base is lithium diisopropylamide. In some embodiments, the base is LiHMDS. In some embodiments, the base is NaHMDS. In some embodiments, the base is KHMDS. In some embodiments, the base is t-BuOK. In some embodiments, the base is t-BuONa. In some embodiments, the base is t-BuOLi. In some embodiments, the base is NaH. In some embodiments, the solvent is selected from tetrahydrofuran, 2-methyltetrahydrofuran, dioxane, and toluene. In some embodiments, the solvent is tetrahydrofuran. In some embodiments, the solvent is 2-methyltetrahydrofuran. In some embodiments, the solvent is dioxane. In some embodiments, the solvent is toluene.

In some embodiments of a process for the preparation of (3,5-dibromo-4-hydroxyphenyl)(2-(1-hydroxyethyl)benzofuran-3-yl-4,5,6,7-d₄)methanone (Compound 1), the compound with the structure:

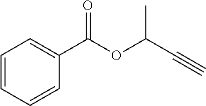

is prepared by a process comprising contacting a compound with the structure:

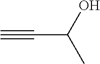

with benzoyl chloride and a base in the presence of a solvent. In some embodiments, the base is selected from pyridine, potassium carbonate, sodium hydroxide, triethylamine, diisopropylethylamine, 1,8-diazabicyclo(5.4.0)undec-7-ene, 1,4-diazabicyclo[2.2.2]octane, and lutidine. In some embodiments, the base is pyridine. In some embodiments, the base is potassium carbonate. In some embodiments, the base is sodium hydroxide. In some embodiments, the base is triethylamine. In some embodiments, the base is diisopropylethylamine. In some embodiments, the base is 1,8-diazabicyclo(5.4.0)undec-7-ene. In some embodiments, the base is 1,4-diazabicyclo[2.2.2]octane. In some embodiments, the base is lutidine. In some embodiments, the solvent is selected from dichloromethane, tetrahydrofuran, 2-methyltetrahydrofuran, acetonitrile, dimethylformamide, dimethyl sulfoxide, and N-methyl-2-pyrrolidone. In some embodiments, the solvent is dichloromethane. In some embodiments, the solvent is tetrahydrofuran. In some embodiments, the solvent is 2-methyltetrahydrofuran. In some embodiments, the solvent is acetonitrile. In some embodiments, the solvent is dimethylformamide. In some embodiments, the solvent is dimethyl sulfoxide. In some embodiments, the solvent is N-methyl-2-pyrrolidone.

In some embodiments of a process for the preparation of (3,5-dibromo-4-hydroxyphenyl)(2-(1-hydroxyethyl)benzofuran-3-yl-4,5,6,7-d₄)methanone (Compound 1), the compound with the structure:

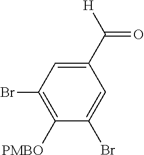

is prepared by a process comprising contacting a compound with the structure:

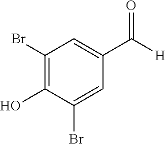 [SMILES: O=Cc1cc(Br)c(O)c(Br)c1]

with 4-methoxybenzyl chloride and a base in the presence of a solvent. In some embodiments, the base is selected from potassium carbonate, sodium hydride, sodium carbonate, sodium hydroxide, potassium hydroxide, lithium hydroxide, triethylamine, diisopropylethylamine, and pyridine. In some embodiments, the base is potassium carbonate. In some embodiments, the base is sodium hydride. In some embodiments, the base is sodium carbonate. In some embodiments, the base is sodium hydroxide. In some embodiments, the base is potassium hydroxide. In some embodiments, the base is lithium hydroxide. In some embodiments, the base is triethylamine. In some embodiments, the base is diisopropylethylamine. In some embodiments, the base is pyridine. In some embodiments, the solvent is selected from acetonitrile, dichloromethane, chloroform, tetrahydrofuran, 2-methyltetrahydrofuran, dimethylformamide, dimethyl sulfoxide, and N-methyl-2-pyrrolidone. In some embodiments, the solvent is acetonitrile. In some embodiments, the solvent is dichloromethane. In some embodiments, the solvent is chloroform. In some embodiments, the solvent is tetrahydrofuran. In some embodiments, the solvent is 2-methyltetrahydrofuran. In some embodiments, the solvent is dimethylformamide. In some embodiments, the solvent is dimethyl sulfoxide. In some embodiments, the solvent is N-methyl-2-pyrrolidone.

In some embodiments of a process for the preparation of (3,5-dibromo-4-hydroxyphenyl)(2-(1-hydroxyethyl)benzofuran-3-yl-4,5,6,7-d₄)methanone (Compound 1), the compound with the structure:

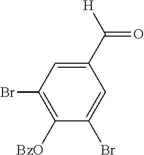

is prepared by a process comprising contacting a compound with the structure:

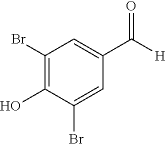 [SMILES: O=Cc1cc(Br)c(O)c(Br)c1]

with benzoyl chloride and a base in the presence of a solvent. In some embodiments, the base is selected from potassium carbonate, sodium hydride, sodium carbonate, sodium hydroxide, potassium hydroxide, lithium hydroxide, triethylamine, diisopropylethylamine, and pyridine. In some embodiments, the base is potassium carbonate. In some embodiments, the base is sodium hydride. In some embodiments, the base is sodium carbonate. In some embodiments, the base is sodium hydroxide. In some embodiments, the base is potassium hydroxide. In some embodiments, the base is lithium hydroxide. In some embodiments, the base is triethylamine. In some embodiments, the base is diisopropylethylamine. In some embodiments, the base is pyridine. In some embodiments, the solvent is selected from acetonitrile, dichloromethane, chloroform, tetrahydrofuran, 2-methyltetrahydrofuran, dimethylformamide, dimethyl sulfoxide, and N-methyl-2-pyrrolidone. In some embodiments, the solvent is acetonitrile. In some embodiments, the solvent is dichloromethane. In some embodiments, the solvent is chloroform. In some embodiments, the solvent is tetrahydrofuran. In some embodiments, the solvent is 2-methyltetrahydrofuran. In some embodiments, the solvent is dimethylformamide. In some embodiments, the solvent is dimethyl sulfoxide. In some embodiments, the solvent is N-methyl-2-pyrrolidone.

In some embodiments of a process for the preparation of (3,5-dibromo-4-hydroxyphenyl)(2-(1-hydroxyethyl)benzofuran-3-yl-4,5,6,7-d₄)methanone (Compound 1), the compound with the structure:

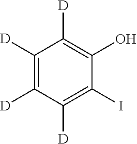 [SMILES: [2H]c1c([2H])c([2H])c(I)c(O)c1[2H]]

is prepared by a process comprising contacting a compound with the structure:

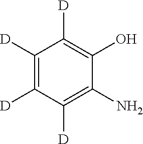 [SMILES: [2H]c1c([2H])c([2H])c(O)c(N)c1[2H]]

with sodium nitrite, potassium iodide, and toluenesulfonic acid.

In some embodiments of a process for the preparation of (3,5-dibromo-4-hydroxyphenyl)(2-(1-hydroxyethyl)benzofuran-3-yl-4,5,6,7-d₄)methanone (Compound 1), the compound with the structure:

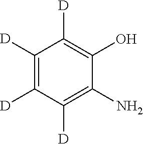 [SMILES: [2H]c1c([2H])c([2H])c(O)c(N)c1[2H]]

is prepared by a process comprising contacting a compound with the structure:

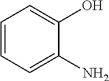

with 10% platinum on carbon and deuterium oxide.

Further disclosed herein, is a process for the preparation of (3,5-dibromo-4-hydroxyphenyl)(2-(1-hydroxyethyl)benzofuran-3-yl-4,5,6,7-d₄)methanone (Compound 1), comprising:
A) the reaction of a compound with the structure:

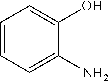

with 10% platinum on carbon and deuterium oxide to produce a compound with the structure:

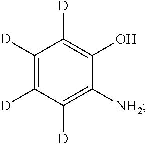 [SMILES: [2H]c1c([2H])c([2H])c(O)c(N)c1[2H]]

B) followed by the reaction of the compound with the structure:

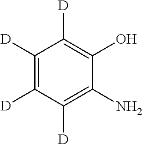 [SMILES: [2H]c1c([2H])c([2H])c(O)c(N)c1[2H]]

with sodium nitrite, potassium iodide, and toluenesulfonic acid to produce a compound with the structure:

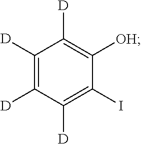 [SMILES: [2H]c1c([2H])c([2H])c(I)c(O)c1[2H]]

C) the reaction of the compound with the structure:

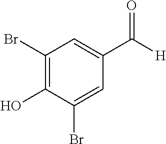 [SMILES: O=Cc1cc(Br)c(O)c(Br)c1]

with 4-methoxybenzyl chloride and potassium carbonate to produce a compound with the structure:

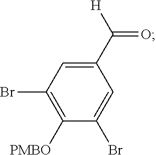

D) the reaction of the compound with the structure:

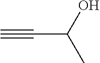

with benzoyl chloride and pyridine to produce a compound with the structure:

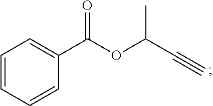

E) followed by the reaction of the compounds with the structure:

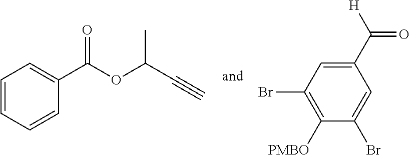

with lithium diisopropylamide to produce a compound with the structure:

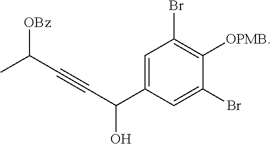

F) followed by the reaction of the compound with the structure:

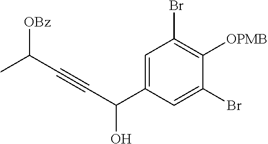

with sodium bicarbonate, potassium bromide, (2,2,6,6-tetramethylpiperidin-1-yl)oxyl (TEMPO), and sodium hypochlorite to produce a compound with the structure:

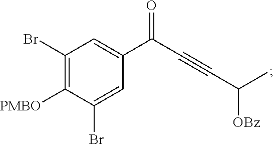

G) followed by the reaction of the compounds with the structures:

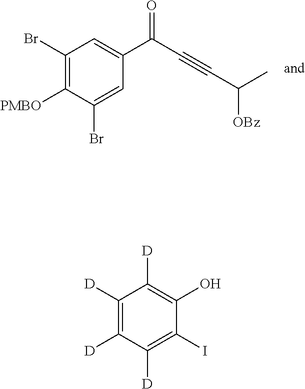

and potassium phosphate followed by contact with trifluoroacetic acid to produce a compound with the structure:

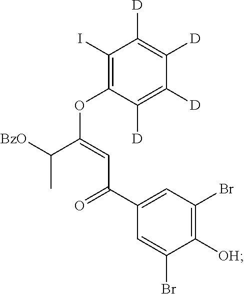

H) followed by the reaction of the compound with the structure:

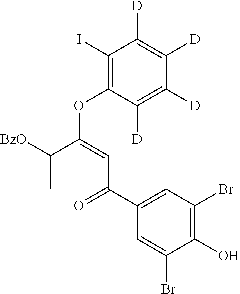

with potassium carbonate and Pd(dppf)Cl₂ to produce a compound with the structure:

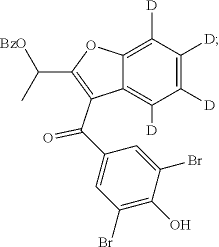

I) followed by the reaction of the compound with the structure:

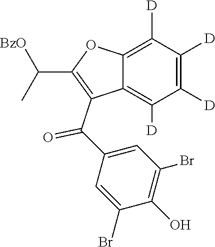

with cesium carbonate to produce a compound with the structure:

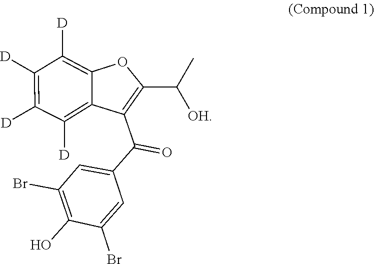 [SMILES: [2H]c1c([2H])c([2H])c2c(C(=O)c3cc(Br)c(O)c(Br)c3)c(C(C)O)oc2c1[2H]]

Further disclosed herein, is a process for the preparation of (3,5-dibromo-4-hydroxyphenyl)(2-(1-hydroxyethyl)benzofuran-3-yl-4,5,6,7-d₄)methanone (Compound 1), comprising:
A) the reaction of a compound with the structure:

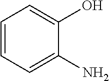

with 10% platinum on carbon and deuterium oxide to produce a compound with the structure:

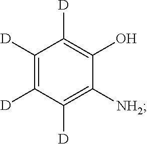 [SMILES: [2H]c1c([2H])c([2H])c(O)c(N)c1[2H]]

B) followed by the reaction of the compound with the structure:

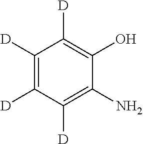 [SMILES: [2H]c1c([2H])c([2H])c(O)c(N)c1[2H]]

with sodium nitrite, potassium iodide, and toluenesulfonic acid to produce a compound with the structure:

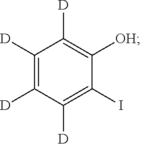 [SMILES: [2H]c1c([2H])c([2H])c(I)c(O)c1[2H]]

C) the reaction of the compound with the structure:

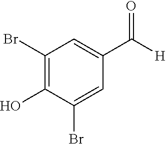 [SMILES: O=Cc1cc(Br)c(O)c(Br)c1]

with benzoyl chloride and potassium carbonate to produce a compound with the structure:

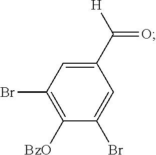

D) the reaction of the compound with the structure:

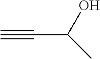

with benzoyl chloride and pyridine to produce a compound with the structure:

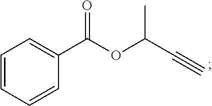

E) followed by the reaction of the compounds with the structure:

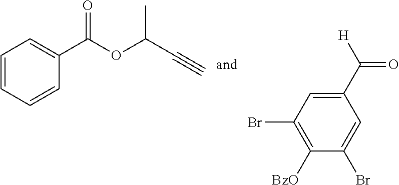

with lithium diisopropylamide to produce a compound with the structure:

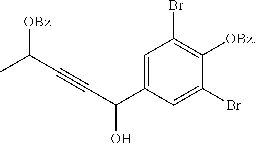

F) followed by the reaction of the compound with the structure:

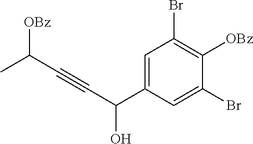

with sodium bicarbonate, potassium bromide, (2,2,6,6-tetramethylpiperidin-1-yl)oxyl (TEMPO), and sodium hypochlorite to produce a compound with the structure:

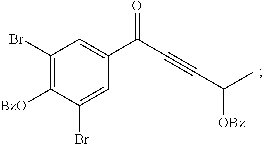

G) followed by the reaction of the compounds with the structures:

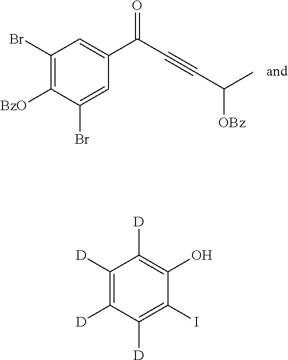

and potassium phosphate to produce a compound with the structure:

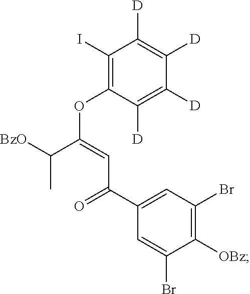

H) followed by the reaction of the compound with the structure:

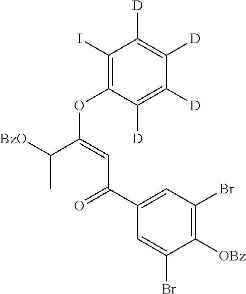

with triethylamine and Pd(dppf)Cl₂ to produce a compound with the structure:

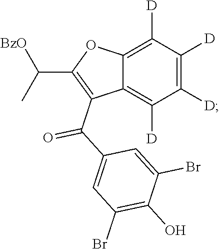

I) followed by the reaction of the compound with the structure:

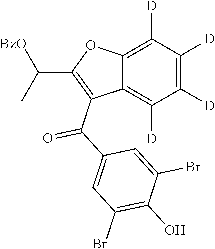

with cesium carbonate to produce a compound with the structure:

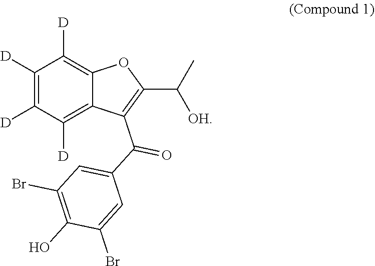 [SMILES: [2H]c1c([2H])c([2H])c2c(C(=O)c3cc(Br)c(O)c(Br)c3)c(C(C)O)oc2c1[2H]]

### Pharmaceutical Compositions and Methods of Administration

Administration of Compound 1 described herein can be in any pharmacological form including a therapeutically effective amount of Compound 1 alone or in combination with a pharmaceutically acceptable carrier.

Pharmaceutical compositions may be formulated in a conventional manner using one or more physiologically acceptable carriers including excipients and auxiliaries which facilitate processing of the active compounds into preparations which can be used pharmaceutically. Proper formulation is dependent upon the route of administration chosen. Additional details about suitable excipients for pharmaceutical compositions described herein may be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference for such disclosure.

A pharmaceutical composition, as used herein, refers to a mixture of Compound 1 described herein, with other chemical components, such as carriers, stabilizers, diluents, dispersing agents, suspending agents, thickening agents, and/or excipients. The pharmaceutical composition facilitates administration of the compound to an organism. In practicing the methods of treatment or use provided herein, therapeutically effective amounts of compounds described herein are administered in a pharmaceutical composition to a mammal having a disease, disorder, or condition to be treated. In some embodiments, the mammal is a human. A therapeutically effective amount can vary widely depending on the severity of the disease, the age and relative health of the subject, the potency of the compound used and other factors. Compound 1 can be used singly or in combination with one or more therapeutic agents as components of mixtures (as in combination therapy).

The pharmaceutical formulations described herein can be administered to a subject by multiple administration routes, including but not limited to, oral, parenteral (e.g., intravenous, subcutaneous, intramuscular), intranasal, buccal, topical, rectal, or transdermal administration routes. Moreover, the pharmaceutical compositions described herein, which include Compound 1 described herein, can be formulated into any suitable dosage form, including but not limited to, aqueous oral dispersions, liquids, gels, syrups, elixirs, slurries, suspensions, aerosols, controlled release formulations, fast melt formulations, effervescent formulations, lyophilized formulations, tablets, powders, pills, dragees, capsules, delayed release formulations, extended release formulations, pulsatile release formulations, multiparticulate formulations, and mixed immediate release and controlled release formulations.

In some embodiments, Compound 1 is formulated in a tablet dosage form. In some embodiments, Compound 1 is formulated in a capsule dosage form. In some embodiments, Compound 1 is formulated in a suspension dosage form. In some embodiments, Compound 1 is formulated as powder-in-capsule dosage form. In some embodiments, Compound 1 is formulated as a powder-in-bottle for reconstitution as a suspension.

Pharmaceutical compositions including a compound described herein may be manufactured in a conventional manner, such as, by way of example only, by means of conventional mixing, dissolving, granulating, dragee-making, levigating, emulsifying, encapsulating, entrapping or compression processes.

Dose administration can be repeated depending upon the pharmacokinetic parameters of the dosage formulation and the route of administration used.

It is especially advantageous to formulate compositions in dosage unit form for ease of administration and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms are dictated by and directly dependent on (a) the unique characteristics of Compound 1 and the particular therapeutic effect to be achieved and (b) the limitations inherent in the art of compounding such an active compound for the treatment of sensitivity in individuals. The specific dose can be readily calculated by one of ordinary skill in the art, e.g., according to the approximate body weight or body surface area of the patient or the volume of body space to be occupied. The dose will also be calculated dependent upon the particular route of administration selected. Further refinement of the calculations necessary to determine the appropriate dosage for treatment is routinely made by those of ordinary skill in the art. Exact dosages are determined in conjunction with standard dose-response studies. It will be understood that the amount of the composition actually administered will be determined by a practitioner, in the light of the relevant circumstances including the condition or conditions to be treated, the choice of composition to be administered, the age, weight, and response of the individual patient, the severity of the patient's symptoms, and the chosen route of administration.

### EXAMPLES

All chemicals, reagents, and solvents were purchased from commercial sources when available and used without further purification.

### Example 1: Synthesis of (3,5-dibromo-4-hydroxyphenyl)(2-(1-hydroxyethyl)benzofuran-3-yl-4,5,6,7-d₄)methanone (Compound 1)

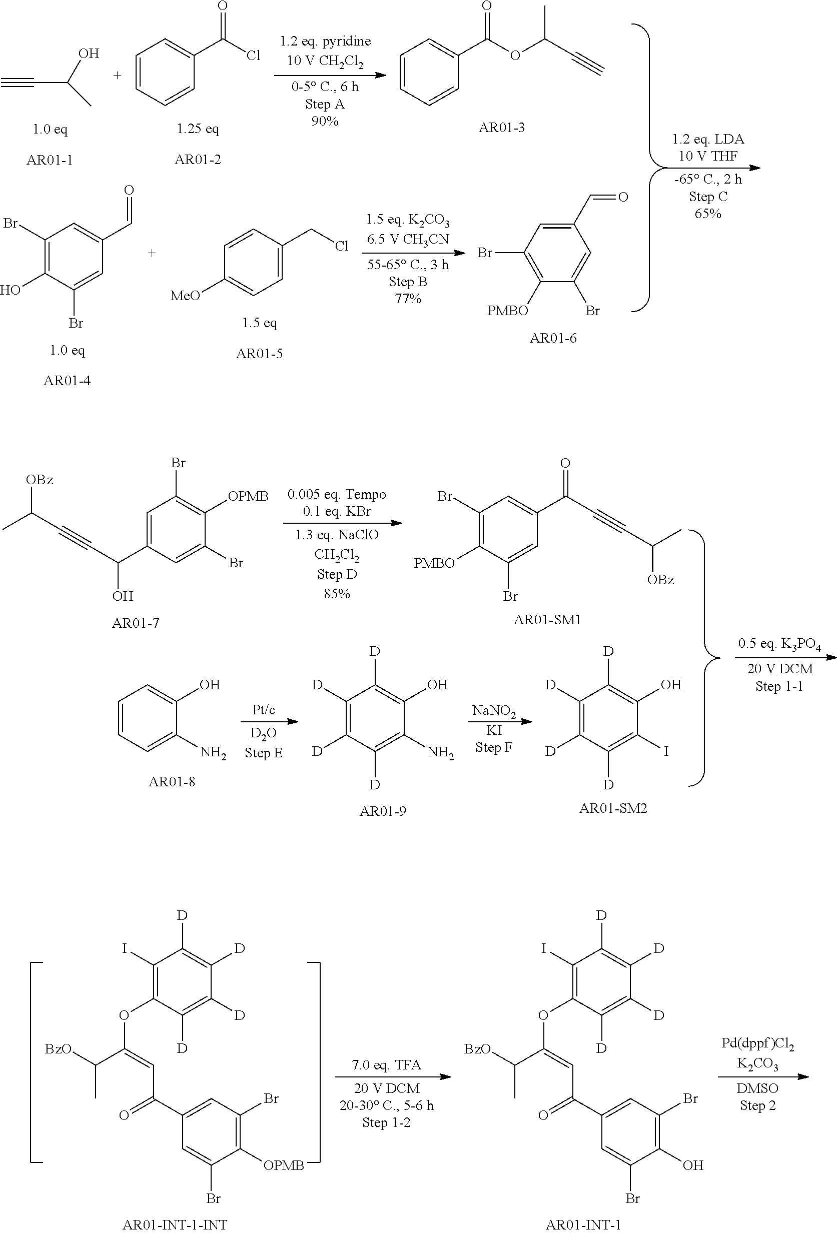

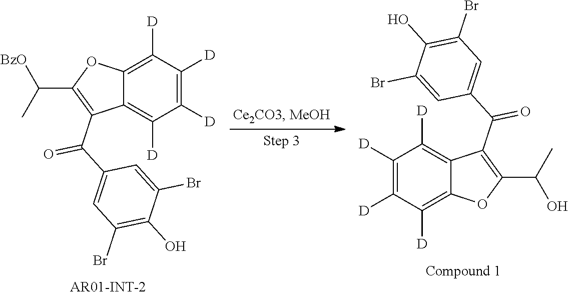

Step A process description:
1) Charged AR01-1 (712 g, 1.0 eq.), DCM (4300 mL) into a flask under N₂
2) Cooled to 0-10° C.
3) Charged AR01-2 (1570 g, 1.1 eq.) dropwise into the mixture at 0-10° C.
4) Stirred the mixture for 0.5 h at 0-10° C.
5) Warmed to 15-25° C.
6) Stirred the mixture at 15-25° C. for 1 h
7) Pour the reaction solution into ice water
8) Charged HCl dropwise slowly into the mixture and stir
9) Liquid separation organic phase wash with NaHCO₃
10) Dried with Na₂SO₄
11) Concentrated under reduced pressure
12) Reduce pressure distillation gave AR01-3 (1595 g, 90.1%) as a white solid. ¹H NMR (400 MHz, DMSO) δ 7.98 (d, J=7.8 Hz, 2H), 7.70 (t, J=7.4 Hz, 1H), 7.56 (t, J=7.6 Hz, 2H), 5.73-5.53 (m, 1H), 3.61 (d, J=1.6 Hz, 1H), 1.57 (d, J=6.7 Hz, 3H).

Step B process description:
1) Charged AR01-4 (2000 g, 1.0 eq.), K₂CO₃ (1185 g, 1.5 eq.), CH₃CN (13 L) into a 20 L flask under N2
2) Stirred the mixture for 0.5 h at 20-30° C.
3) Charged AR01-5 (1296 g, 1.5 eq.) dropwise into the mixture
4) Charged NaI into the flask
5) Warmed to 58-68° C.
6) Stirred the mixture for 5 h at 55-65° C.
7) Filtrated
8) Concentrated the mixture under vacuo to obtain AR01-6
9) Recrystallization gave AR01-6 as a white solid (1670 g, 77.3%) as a white solid. ¹H NMR (400 MHz, DMSO) δ 9.92 (s, 1H), 8.20 (s, 2H), 7.50 (d, J=8.4 Hz, 2H), 6.99 (d, J=8.4 Hz, 2H), 5.03 (s, 2H), 3.79 (s, 3H).

Step C process description:
1) Charged AR01-3 (727 g, 1.0 eq.), THE (10 L) into a flask under N2
2) Cooled down to −70→−60° C.
3) Charged LDA (2.5 L) dropwise into the mixture
4) Stirred the mixture at −70→−60° C. for 30 min
5) Charged AR01-6 (1670 g, 1.0 eq.), THE (3.4 L) into the mixture under N2 at −70→−60° C.
6) Stirred the mixture at −70→−60° C. for 1.5 h
7) Charged AcOH (601 g), NH₄Cl aq.
8) Separated the water layer
9) Dried with Na₂SO₄
10) Concentrated under reduced pressure
11) Purification by column chromatography gave AR01-7 (4.1 kg, 65%) as a yellow solid. ¹H NMR (400 MHz, DMSO) δ 7.99 (d, J=7.4 Hz, 2H), 7.71 (s, 2H), 7.68 (d, J=7.4 Hz, 1H), 7.55 (t, J=7.4 Hz, 2H), 7.48 (d, J=8.1 Hz, 2H), 6.97 (d, J=7.9 Hz, 2H), 6.33 (s, 1H), 5.71 (d, J=6.8 Hz, 1H), 4.89 (s, 2H), 3.77 (s, 3H), 1.59 (d, J=6.5 Hz, 3H).

Step D process description:
1) Charged AR01-7 (4100 g), DCM (24.6 mL), H₂O (16.4 L), KBr (85 g), NaHCO₃ (600 g) into a flask
2) Cooled down to 0-5° C.
3) 3° C., Charge Tempo (56 g) into the mixture
4) Charged NaOCl aq. (7250 g) dropwise into the mixture at 0-5° C.
5) Liquid separation
6) The aqueous phase was extracted with DCM
7) The organic layer washed with saturated Na₂S₂O₃ aq. and water
8) Dried over Na₂SO₄ and concentrated under reduced pressure to give crude AR01-SM1.
9) Recrystallization from CH₃CN gave AR01-SM1 (2.5 kg, 61.2%) as a white solid. ¹H NMR (400 MHz, DMSO) δ 8.23 (s, 2H), 8.08-8.02 (m, 2H), 7.72 (t, J=7.4 Hz, 1H), 7.59 (t, J=7.7 Hz, 2H), 7.48 (d, J=8.6 Hz, 2H), 6.98 (d, J=8.6 Hz, 2H), 5.94 (q, J=6.8 Hz, 1H), 5.02 (s, 2H), 3.78 (s, 3H), 1.74 (d, J=6.8 Hz, 3H).

Step E process description:
1) To a 2000 mL flask was added AR01-8 (30 g, 1.0 eq.), D₂O (600 mL) and 10% Pt/C (3 g)
2) Degassed the flask with N₂ three times, and then degassed with H₂ three times
3) Heated the mixture to 120° C. and the mixture was stirred at 120° C. for 48 h
4) The mixture was cooled to 25-35° C. and EA (600 mL) was added
5) Filtered and washed the pad with EA (60 mL)
6) Separated the bi-phase and extracted the aqueous layer with EA (300 mL) twice
7) The combined organic layer was concentrated to 2-3 V under vacuum at 40-45° C.
8) The mixture was concentrated till no distillate produced under vacuum at 40-45° C.
9) D₂O (600 mL) was added to the residuals
10) 10% Pt/C (3 g) was added and degassed the flask with N₂ three times, and then degassed with H₂ three times
11) Heated the mixture to 120° C. and the mixture was stirred at 120° C. for 24 h.
12) The mixture was cooled to 25-35° C. and EA (600 mL) was added
13) Filtered and washed the pad with EA (60 mL)
14) Separated the bi-phase and extracted the aqueous layer with EA (300 mL) twice
15) The combined organic layer was concentrated to 2-3 V under vacuum at 40-45° C.
16) Adjusted the temperature to 30-40° C. and n-heptane (240 mL, 8 V) was added slowly
17) The mixture was cooled to 0-5° C. slowly and stirred at 0-5° C. for 0.5-1 h
18) Dried the wet product under vacuum at 40-45° C.
19) Obtained AR01-9 (25 g, 83.0% yield) as a bronze solid. ¹H NMR (400 MHz, DMSO) δ
9.36 (s, 1H), 6.74 (t, 1H), 6.72 (t, 1H), 6.62 (d, 1H), 6.60 (d, 1H).

Step F process description:
1) To a 5000 mL flask was added AR01-9 (100.0 g, 1.0 eq.) and ACN (1000 mL), H2O (600 mL)
2) To the mixture was added TsOH (3.0 eq.) and the mixture was stirred for 0.5 h
3) Cooled the mixture to 0-5° C. and then NaNO₂ (1.3 eq.) in H₂O (200 mL) solution was added slowly
4) The mixture was stirred at 0-5° C. for 0.5-1 h
5) Adjusted the temperature to −5-0° C. and KI (2.0 eq.) in H₂O (200 mL) solution was added slowly over 30 min
6) Adjusted the temperature to 0-30° C. and stirred the mixture for 8-10 h
7) Sat. Na₂SO₃ aqueous solution was added and the mixture was stirred at 0-15° C. for 1 h
8) Separated the mixture and extracted the mixture with EA (1000 mL) twice
9) Washed the organic layer with sat NaCl solution (500 mL)
10) Filtered the organic layer by a silicon pad
11) The combined organic layer was concentrated to 2-3 V under vacuum at 35-45° C.
12) N-heptane (500 mL) was added to the mixture
13) The mixture was concentrated to 2-3 V under vacuum at 35-45° C.
14) To the mixture was added n-heptane (500 mL)
15) The mixture was cooled to −10-0° C. slowly and stirred at −10-0° C. for 0.5-1 h
16) Filtered and the wet cake was washed with cooled n-heptane (1-2 v)
17) Dried the wet product under vacuum at 25-30° C.
18) Obtained AR01-SM2 (110 g, 55.6% yield)¹H NMR (400 MHz, DMSO) δ 10.26 (s, 1H), 7.66 (s, 1H), 7.19 (s, 1H), 6.89 (s, 1H), 6.59 (s, 1H).

Step 1 process description:
1) A 1 L three-necked flask was added AR01-SM1 (25.75 g, 1.0 eq.), potassium phosphate (4.8 g, 1.0 eq.), DCM (460 ml, 18 V)
2) Added AR01-SM2 (10.0 g, 1.0 eq.), DCM (50 ml, 2 V) to the 100 ml constant pressure dropping funnel, and the system was protected by nitrogen
3) The internal temperature drops to 5 to 10° C., and the DCM solution of AR01-SM2 was added dropwise
4) After dripping, kept the inner temperature at 5 to 10° C. and reacted for 2 h
5) Added TFA (25.65 g, 5.0 eq.) dropwise to the reaction solution raise the internal temperature of the reaction solution to 25 to 30° C. and reacted for 2 h
6) Slowly added NaHCO₃ aq. dropwise to the reaction solution, adjusted pH=7-8, added water (125 mL, 5 V), continued to stir for 30 min, separated the layers, extracted the aqueous phase with DCM (50 mL, 2 V), combined the organic phases, and washed water 125 mL*4 times, dried with anhydrous magnesium sulfate, 40° C. under reduced pressure and concentrated to dryness, added toluene (75 mL, 3 V), n-heptane (26 mL, 1 V) crystals, stirred at room temperature for 2 h, cooled to 0 to 5° C., filtered. The filter cake was washed once with toluene:n-heptane=5:1 (2 V), and the solid was dried to constant weight at 50° C. by blowing air to obtain AR01-INT-1 (25.1 g, 82.5% yield). HPLC m/z calculated for (C₂₄H₁₃D₄Br₂IO₅) [M+H]⁺ 675.5, 677.4, found: 675.5, 677.4. ¹H NMR (400 MHz, DMSO) δ 10.99 (s, 1H), 8.33-7.86 (m, 2H), 7.77 (s, 2H), 7.67 (t, J=7.4 Hz, 1H), 7.54 (t, J=7.7 Hz, 2H), 6.50 (q, J=6.6 Hz, 1H), 5.74 (s, 1H), 1.79 (d, J=6.6 Hz, 3H).

Step 2 process description:
1) Added AR01-INT-1 (20.0 g, 1.0 eq.), K₂CO₃ (12.3 g, 3.0 eq.), Pd(dppf)Cl₂ (1.08 g, 0.05 eq.), DMSO (400 mL, 20 V) to a 1 L three-necked flask
2) Turned on magnetic stirring, replaced with N₂ three times, and protect with N₂
3) The internal temperature was controlled at 90-95° C. to react for 3 h
4) The reaction solution was cooled to room temperature, added EA (400 mL, 20 V), mixed well, slowly added dropwise to HCl aq. and water (200 ml, 10 V), adjusted pH=2-3, continued to stir for 2 h, filtered (padded diatomaceous earth), the filter cake was washed with EA (80 mL, 4 V), the filtrate was separated into layers, the aqueous phase was washed with EA (80 mL, 4 V), the organic phases were combined, washed with water 100 ml*4 times, concentrated the organic phases under reduced pressure at 45° C. to dryness, added acetone (80 ml, 4 V), filtered, washed filter cake with ice acetone (20 mL, 1 V), the solid was dried under reduced pressure at 50° C. to constant weight to obtain AR01-INT-2 (12.1 g, 74.6% yield). HPLC m/z calculated for (C₂₄H₁₂D₄Br₂O₅) [M−H]⁻ 545.5, 547.6 found: 545.5, 547.6. ¹H NMR (400 MHz, DMSO) δ 11.11 (s, 1H), 7.92 (s, 2H), 7.90-7.86 (m, 2H), 7.65 (dd, J=10.6, 4.3 Hz, 1H), 7.49 (t, J=7.8 Hz, 2H), 6.17 (q, J=6.6 Hz, 1H), 1.78 (d, J=6.7 Hz, 3H).

Step 3 process description:
1) Charged AR01-INT-2 (14.31 g, 1.0 eq.) and MeOH (10 V) to reactor
2) Charged Cs₂CO₃ (25.5 g, 3.0 eq.) by batches at 28±5° C.
3) Stirred for at least 16 h at 28±5° C.
4) Charged purified water (280 mL, 20 V) to reaction at 25±5° C.
5) Adjusted pH to 2˜3 with 12 N HCl aq. at 25±5° C. Stirred for at least 1 h
6) Filtered and washed with purified water (28 mL, 2 V) twice
7) Dissolved the filter cake with EA (215 mL, 15 V). Charged 1 N HCl solution (70 mL, 5 V)
8) Stirred for at least 1 h and separated
9) Washed organic phase with 20% NaCl aqueous (70 mL, 5 V) for once
10) Concentrated to 2 V˜3 V under vacuum at 40±5° C. and charged EA (84 ml, 6 V)
11) Concentrated to 2 V˜3 V under vacuum at 40±5° C. and charged EA (84 ml, 6 V)
12) Concentrated to 2 V˜3 V under vacuum at 40±5° C.
13) Charged EA (140 mL, 10V), added activated carbon, increased the internal temperature to 50±5° C., stirred for one hour, filtered and washed the filter cake with EA (28 ml, 2V)
14) Concentrated to 2 V˜3 V under vacuum at 40±5° C.
15) Charged n-heptane (140 mL, 10 V) dropwise and stirred for at least 2 h at 50±5° C.
16) Cooled to 25±5° C. and stirred for at least 12 h
17) Filtered and washed with n-heptane (14 mL, 1 V) twice
18) Dried for at least 12 h at 50±5° C. under vacuum to give Compound 1 (8.7 g, 81% yield) as an off-white solid. HPLC m/z calculated for (C₁₇H₈D₄Br₂O₄) [M+H]⁺ 425.3, 427.3, found: 425.3, 427.3. ¹H NMR (400 MHz, DMSO) δ 11.08 (s, 1H), 7.95 (s, 2H), 5.59 (s, 1H), 4.86 (d, J=6.6 Hz, 1H), 1.48 (d, J=6.6 Hz, 3H).

### Example 2: Alternative synthesis of (3,5-dibromo-4-hydroxyphenyl)(2-(1-hydroxyethyl)benzofuran-3-yl-4,5,6,7-d₄)methanone (Compound 1)

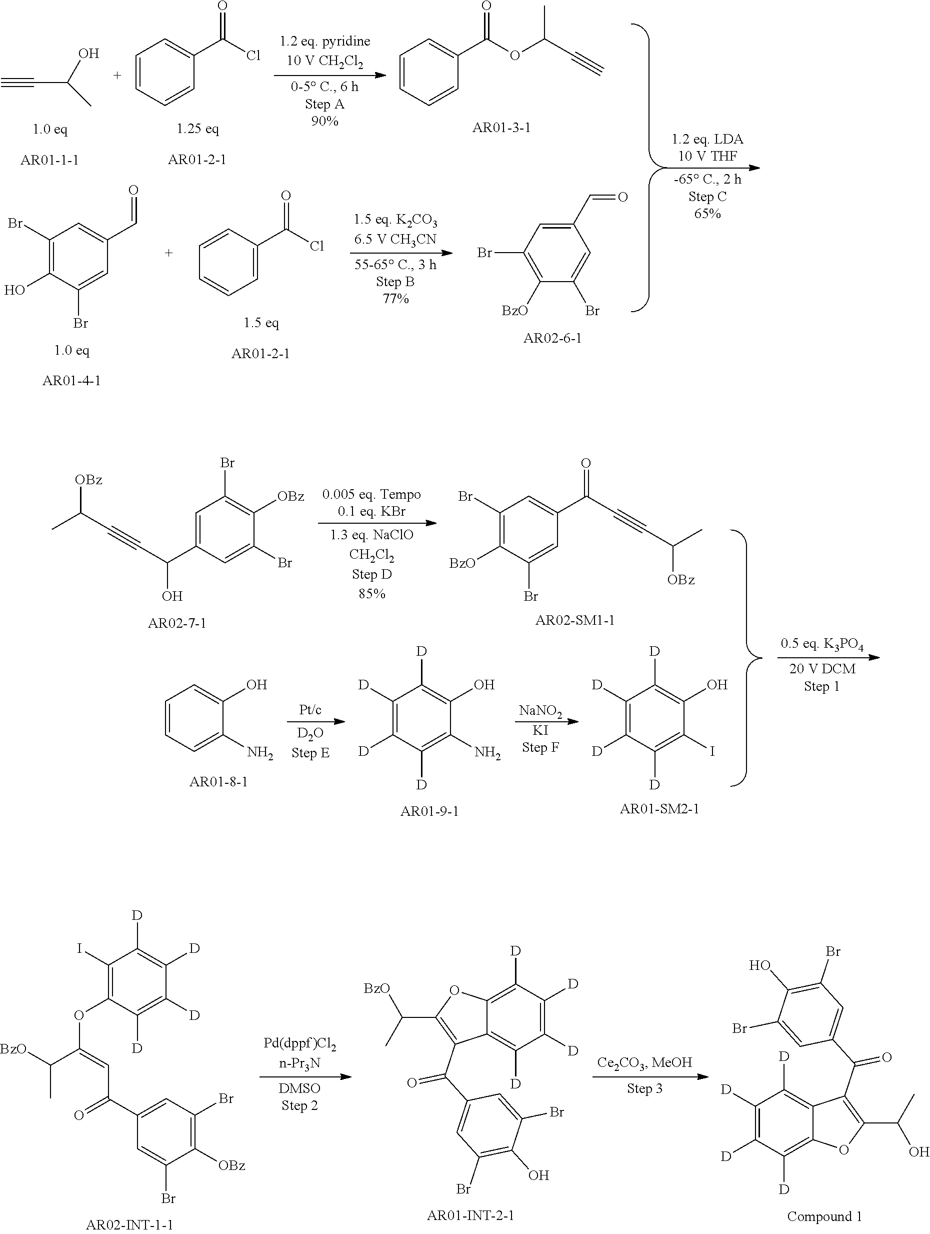

Steps A, E, F, and 3 were completed as described in Example 1. Steps B, C, D, 1, and 2 were completed as described below.

Step B process description:
1) Charged AR01-4-1 (2000 g, 1.0 eq.), K₂CO₃ (1185 g, 1.5 eq.), CH₃CN (13 L) into a 20 L flask under N₂
2) Stirred the mixture for 0.5 h at 0-10° C.
3) Charged AR01-2-1 (1511.9 g, 1.5 eq.) dropwise into the mixture
4) Stirred the mixture for 5 h at 0-10° C.
5) Filtrated
6) Concentrated the mixture under vacuo to obtain AR02-6-1
7) Recrystallization to obtain AR02-6-1 (2199.5 g, 80%) as a white solid.

Step C process description:
1) Charged AR01-3-1 (727 g, 1.0 eq.), THF (10 L) into a flask under N₂
2) Cooled down to −70→−60° C.
3) Charged LDA (2.5 L) dropwise into the mixture
4) Stirred the mixture at −70→−60° C. for 30 min
5) Charged AR02-6-1 (1595.2 g, 1.0 eq.), THE (3.4 L) into the mixture under N₂ at −70→−60° C.
6) Stirred the mixture at −70→−60° C. for 1.5 h
7) Charged AcOH (601 g), NH₄Cl aq.
8) Separated the water layer
9) Dried with Na₂SO₄
10) Concentrated under reduced pressure
11) Purification by column chromatography to obtain AR02-7-1 (1.51 kg, 65%) as a yellow solid. LCMS (254m, t=1.86 min) calculated for (C₂₅H₁₈Br₂O₅) [M−17]⁻=539.3, 541.3, found: 539.3, 541.3.

Step D process description:
1) Charged AR02-7-1 (3984.5 g), DCM (24.6 L), H₂O (16.4 L), KBr (85 g), NaHCO₃ (600 g) into a flask
2) Cooled to 0˜5° C.
3) 3° C., Charge Tempo (56 g) into the mixture
4) Charged NaOCl aq. (7250 g) dropwise into the mixture at 0˜5° C.
5) Liquid separation
6) The aqueous phase was extracted with DCM
7) The organic layer washed with saturated Na₂S₂O₃ aq. and water
8) Dried over Na₂SO₄ and concentrated under reduced pressure to give crude AR02-SM1-1.
9) Recrystallization from CH₃CN to obtain AR02-SM1-1 (3.26 kg, 82%) as a white solid. 1H NMR (400 MHz,) δ 8.21-8.13 (m, 4H), 8.04 (dd, J=8.3, 1.3 Hz, 2H), 7.83-7.49 (m, 8H), 5.96 (q, J=6.8 Hz, 1H), 1.75 (d, J=6.8 Hz, 3H).

Step 1 process description:
1) A 1 L three-necked flask was added AR02-SM1-1 (25.75 g, 1.0 eq.), potassium phosphate (4.9 g, 0.5 eq.), DCM (460 ml, 18 V)
2) Added AR02-SM2-1 (10.0 g, 1.0 eq.), DCM (50 ml, 2 V) to the 100 ml constant pressure dropping funnel, and the system was protected by nitrogen
3) The internal temperature drops to 5 to 10° C., and the DCM solution of AR02-SM2-1 was added dropwise
4) After dripping, kept the inner temperature at 5 to 10° C. and reacted for 2 h
5) Slowly added NaHCO₃ aq. dropwise to the reaction solution, adjusted pH=7˜8, added water (125 mL, 5 V), continued to stir for 30 min, separated the layers, extracted the aqueous phase with DCM (50 mL, 2 V), combined the organic phases, and washed water 125 mL*4 times, dried with anhydrous magnesium sulfate, 40° C. under reduced pressure and concentrated to dryness, added toluene (75 mL, 3 V), n-heptane (26 ml, 1 V) crystals, stirred at room temperature for 2 h, cooled to 0 to 5° C., filtered. The filter cake was washed once with toluene:n-heptane=5:1 (2 V), and the solid was dried to constant weight at 50° C. by blowing air to obtain AR02-INT-1-1 (32.5 g, 91%) as a yellow solid.

Step 2 process description:
1) Added AR02-INT-1-1 (20.0 g, 1.0 eq.), n-Pr₃N (11.3 g, 3.0 eq.), Pd(dppf)Cl₂ (1.08 g, 0.05 eq.), DMSO (400 ml, 20 V) to a 1 L three-necked flask
2) Turned on magnetic stirring, replaced with N₂ three times, and protect with N₂
3) The internal temperature was controlled at 100˜115° C. to react for 3 h
4) The reaction solution was cooled to room temperature, added EA (400 mL, 20 V), mixed well, slowly added dropwise to HCl aq. and water (200 mL, 10 V), adjusted pH=2˜3, continued to stir for 2 h, filtered (padded diatomaceous earth), the filter cake was washed with EA (80 mL, 4 V), the filtrate was separated into layers, the aqueous phase was washed with EA (80 mL, 4 V), the organic phases were combined, washed with water 100 mL*4 times, concentrated the organic phases under reduced pressure at 45° C. to dryness, added acetone (80 mL, 4 V), filtered, washed filter cake with ice acetone (20 mL, 1 V), the solid was dried under reduced pressure at 50° C. to constant weight to obtain AR01-INT-2-1. LCMS (254 nm, t=3.29 min) and m/z calculated for (C₂₄H₁₂D₄Br₂O₅) [M−H]⁻=545.5, 547.5, found: 545.5, 547.5. ¹H NMR (400 MHz, DMSO) δ 11.11 (s, 1H), 7.92 (s, 2H), 7.88 (dd, J=8.3, 1.2 Hz, 2H), 7.69-7.62 (m, 1H), 7.50 (t, J=7.8 Hz, 2H), 6.17 (q, J=6.7 Hz, 1H), 1.78 (d, J=6.7 Hz, 3H).

### Example 3: In Vitro Interaction Studies of Compound 1 and Benzbromarone with the Human URAT1 Uptake Transporter

Uptake experiments were performed using MDCKII cells stably expressing the human URAT1 uptake transporter. Cells were cultured at 37±1° C. in an atmosphere of 95:5 air:CO₂ and were plated onto standard 96-well tissue culture plates at the cell number described in Table 1.

TABLE 1

| | | Cell | | Incubation | |
|---|---|---|---|---|---|
| | Control | number/ | Culturing | prior to | |
| Transporter | cell line | well | medium | the assay | Buffer |
| human | Mock- | 1 × 10⁵ | DMEM | 24 h | HBSS |
| URAT1 | transfected | | 4.5 g/L | | w/o Cl⁻ |
| | MDCKII | | glucose | | (pH 7.4) |

DMEM: Dulbecco's Modified Eagle's Medium;
HBSS: Hank's balanced salt solution;
w/o: without

Before the experiment, the medium was removed and the cells were washed twice with 100 μL of HBSS without Cl⁻. Uptake experiments were carried out at 37±1° C. in 50 μL of HBSS without Cl⁻, pH 7.4 containing the probe substrate (20 μM uric acid) and the test article (TA) or solvent. The organic solvent concentration was equal in all wells, and did not exceed 1% (v/v).

Treatment groups are presented in Table 2.

TABLE 2

| Treatment groups in the 96-well plate format | No. of wells |
|---|---|
| TA in assay buffer (0.01, 0.04, 0.12, 0.37, 1.11, 3.33 and | 3 per TA |
| 10.0 μM) in transfected cells | concentration |
| TA in assay buffer (0.01, 0.04, 0.12, 0.37, 1.11, 3.33 and | 3 per TA |
| 10.0 μM) in control cells | concentration |
| 1% DMSO control in transfected cells | 3 |
| 1% DMSO control in control cells | 3 |
| Reference inhibitor in assay buffer with 1% DMSO in | 3 |
| transfected cells | |
| Reference inhibitor in assay buffer with 1% DMSO in | 3 |
| control cells | |

After the experiment, cells were washed twice with 100 μL of ice cold HBSS without Cl⁻ and lysed with 50 μL of 0.1 M NaOH. Radiolabeled probe substrate transport was determined by measuring an aliquot (35 μL) from each well for liquid scintillation counting.

Results: Both test articles (Compound 1 and benzbromarone) were soluble in HBSS buffer at all tested concentrations; the highest tested concentration being 10 μM. Compound 1 inhibited URAT1 mediated uric acid accumulation by 100% at a concentration of 10 μM with an IC₅₀=0.067 μM. Benzbromarone inhibited URAT1 mediated uric acid accumulation by 98% at a concentration of 10 μM with an IC₅₀=0.196 μM.

The examples and embodiments described herein are for illustrative purposes only and in some embodiments, various modifications or changes are to be included within the purview of disclosure and scope of the appended claims.

What is claimed is:

1. A process for the preparation of (3,5-dibromo-4-hydroxyphenyl)(2-(1-hydroxyethyl)benzofuran-3-yl-4,5,6,7-d₄)methanone (Compound 1): 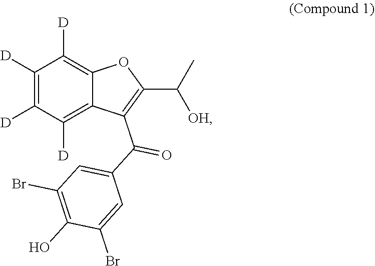 [SMILES: [2H]c1c([2H])c([2H])c2c(C(=O)c3cc(Br)c(O)c(Br)c3)c(C(C)O)oc2c1[2H]]
comprising contacting a compound with the structure: 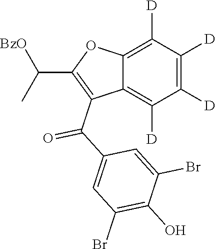
with cesium carbonate in the presence of a solvent.

2. The process of claim 1, wherein the solvent is selected from methanol, ethanol, isopropanol, butanol, water, acetone, acetonitrile, dimethylformamide, dimethyl sulfoxide, N-methyl-2-pyrrolidone, acetic acid, and combinations thereof.

3. The process of claim 1, wherein the solvent is methanol.

4. The process of any one of claims 1-3, wherein the compound with the structure: 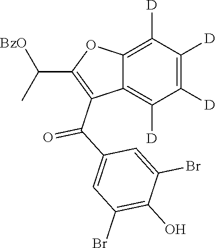
is prepared by a process comprising contacting a compound with the structure: 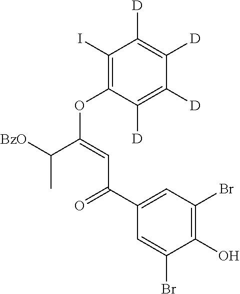
with a base and palladium catalyst in the presence of a solvent.

5. The process of claim 4, wherein the base is selected from potassium carbonate, silver carbonate, sodium carbonate, cesium carbonate, sodium bicarbonate, triethylamine, diisopropylethylamine, 1,8-diazabicyclo(5.4.0)undec-7-ene, and 1,4-diazabicyclo[2.2.2]octane.

6. The process of claim 5, wherein the base is potassium carbonate.

7. The process of any one of claims 4-6, wherein the palladium catalyst is selected from Pd(dppf)Cl₂, PdCl₂, Pd(OAc)₂, Pd(Ph₃P)₄, Pd₂(dba)₃, and Pd/C.

8. The process of any one of claims 4-7, wherein the palladium catalyst is Pd(dppf)Cl₂.

9. The process of any one of claims 4-7, wherein the ligand is selected from Ph₃P, BINAP, DPEphos, S-Phos, Xantphos, dtbpf, and Mephos.

10. The process of any one of claims 4-9, wherein the solvent is selected from dimethyl sulfoxide, dimethylformamide, N-methyl-2-pyrrolidone, acetone, acetonitrile, sulfolane, tetrahydrofuran, and toluene.

11. The process of any one of claims 4-10, wherein the solvent is dimethyl sulfoxide.

12. The process of any one of claims 4-11, wherein the compound with the structure: 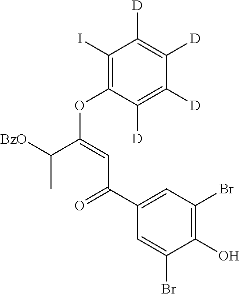
is prepared by a process comprising contacting a compound with the structure: 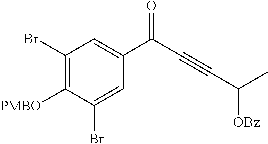
with a compound with the structure: 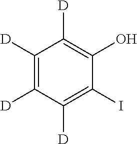 [SMILES: [2H]c1c([2H])c([2H])c(I)c(O)c1[2H]]
and potassium phosphate in the presence of a solvent followed by contact with trifluoroacetic acid.

13. The process of claim 12, wherein the solvent is selected from dichloromethane, chloroform, acetonitrile, toluene, ethyl acetate, and tetrahydrofuran.

14. The process of claim 12 or claim 13, wherein the solvent is dichloromethane.

15. The process of any one of claims 12-14, wherein the compound with the structure: 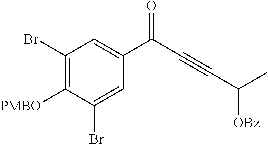
is prepared by a process comprising contacting a compound with the structure: 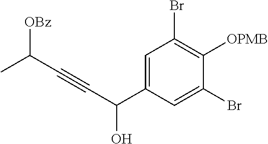
with sodium bicarbonate, potassium bromide, (2,2,6,6-tetramethylpiperidin-1-yl)oxyl (TEMPO), and sodium hypochlorite.

16. The process of claim 15, wherein the compound with the structure: 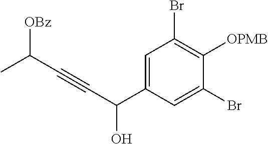
is prepared by a process comprising contacting a compound with the structure: 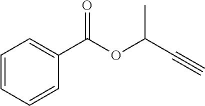
with a compound with the structure: 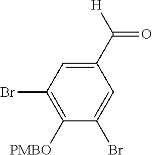
and a base in the presence of a solvent.

17. The process of claim 16, wherein the base is selected from lithium diisopropylamide, LiHMDS, NaHMDS, KHMDS, t-BuOK, t-BuONa, t-BuOLi, and NaH.

18. The process of claim 16 or claim 17, wherein the base is lithium diisopropylamide.

19. The process of any one of claims 16-18, wherein the solvent is selected from tetrahydrofuran, 2-methyltetrahydrofuran, dioxane, and toluene.

20. The process of any one of claims 16-19, wherein the solvent is tetrahydrofuran.

21. The process of any one of claims 16-20, wherein the compound with the structure: 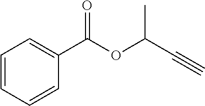
is prepared by a process comprising contacting a compound with the structure: 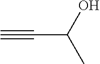
with benzoyl chloride and a base in the presence of a solvent.

22. The process of claim 21, wherein the base is selected from pyridine, potassium carbonate, sodium hydroxide, triethylamine, diisopropylethylamine, 1,8-diazabicyclo(5.4.0)undec-7-ene, and 1,4-diazabicyclo[2.2.2]octane, and lutidine.

23. The process of claim 21 or claim 22, wherein the base is pyridine.

24. The process of any one of claims 21-23, wherein the solvent is selected from dichloromethane, tetrahydrofuran, 2-methyltetrahydrofuran, acetonitrile, dimethylformamide, dimethyl sulfoxide, and N-methyl-2-pyrrolidone.

25. The process of any one of claims 21-24, wherein the solvent is dichloromethane.

26. The process of any one of claims 16-20, wherein the compound with the structure: 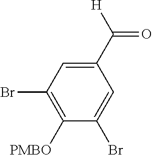
is prepared by a process comprising contacting a compound with the structure: 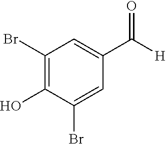 [SMILES: O=Cc1cc(Br)c(O)c(Br)c1]
with 4-methoxybenzyl chloride and a base in the presence of a solvent.

27. The process of claim 26, wherein the base is selected from potassium carbonate, sodium hydride, sodium carbonate, sodium hydroxide, potassium hydroxide, lithium hydroxide, triethylamine, diisopropylethylamine, and pyridine.

28. The process of claim 26 or claim 27, wherein the base is potassium carbonate.

29. The process of any one of claims 26-28, wherein the solvent is selected from acetonitrile, dichloromethane, chloroform, tetrahydrofuran, 2-methyltetrahydrofuran, dimethylformamide, dimethyl sulfoxide, and N-methyl-2-pyrrolidone.

30. The process of any one of claims 26-29, wherein the solvent is acetonitrile.

31. The process of any one of claims 12-14, wherein the compound with the structure: 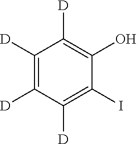 [SMILES: [2H]c1c([2H])c([2H])c(I)c(O)c1[2H]]
is prepared by a process comprising contacting a compound with the structure: 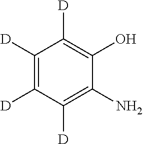 [SMILES: [2H]c1c([2H])c([2H])c(O)c(N)c1[2H]]
with sodium nitrite, potassium iodide, and toluenesulfonic acid.

32. The process of claim 31, wherein the compound with the structure: 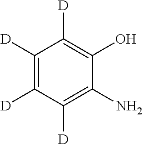 [SMILES: [2H]c1c([2H])c([2H])c(O)c(N)c1[2H]]
is prepared by a process comprising contacting a compound with the structure: 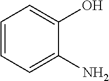
with 10% platinum on carbon and deuterium oxide.

33. A process for the preparation of (3,5-dibromo-4-hydroxyphenyl)(2-(1-hydroxyethyl)benzofuran-3-yl-4,5,6,7-d₄)methanone (Compound 1), comprising:
A) the reaction of a compound with the structure: 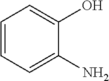
with 10% platinum on carbon and deuterium oxide to produce a compound with the structure: 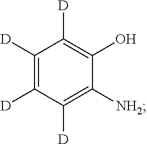 [SMILES: [2H]c1c([2H])c([2H])c(O)c(N)c1[2H]]
B) followed by the reaction of the compound with the structure: 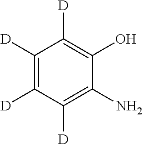 [SMILES: [2H]c1c([2H])c([2H])c(O)c(N)c1[2H]]
with sodium nitrite, potassium iodide, and toluenesulfonic acid to produce a compound with the structure: 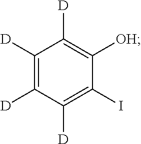 [SMILES: [2H]c1c([2H])c([2H])c(I)c(O)c1[2H]]
C) the reaction of a compound with the structure: 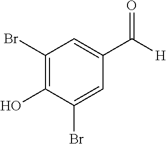 [SMILES: O=Cc1cc(Br)c(O)c(Br)c1]
with 4-methoxybenzyl chloride and potassium carbonate to produce a compound with the structure: 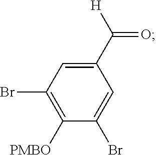
D) the reaction of the compound with the structure: 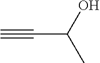
with benzoyl chloride and pyridine to produce a compound with the structure: 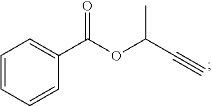
E) followed by the reaction of the compounds with the structures: 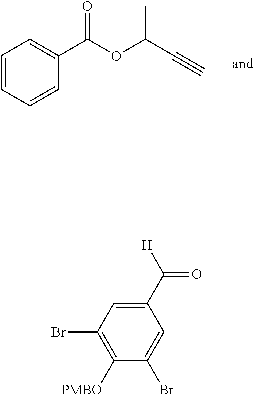
with lithium diisopropylamide to produce a compound with the structure: 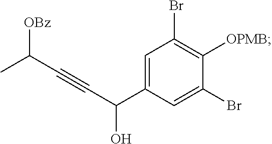
F) followed by the reaction of the compound with the structure: 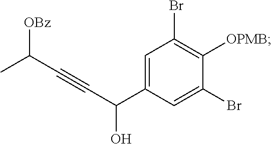
with sodium bicarbonate, potassium bromide, (2,2,6,6-tetramethylpiperidin-1-yl)oxyl (TEMPO), and sodium hypochlorite to produce a compound with the structure: 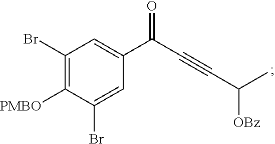
G) followed by the reaction of the compounds with the structures: 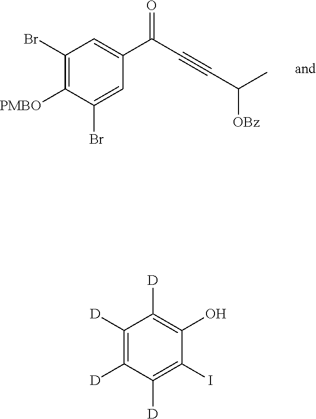
and potassium phosphate, followed by contact with trifluoroacetic acid to produce a compound with the structure: 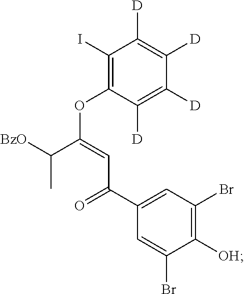
H) followed by the reaction of the compound with the structure: 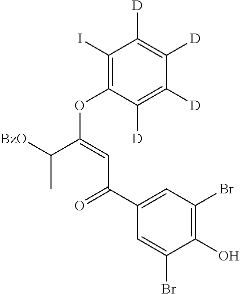
with potassium carbonate and Pd(dppf)Cl₂ to produce a compound with the structure: 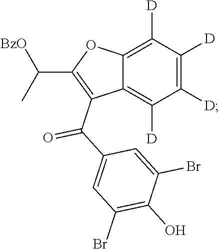
I) followed by the reaction of the compound with the structure: 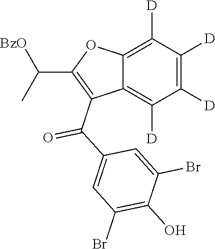
with cesium carbonate to produce a compound with the structure:

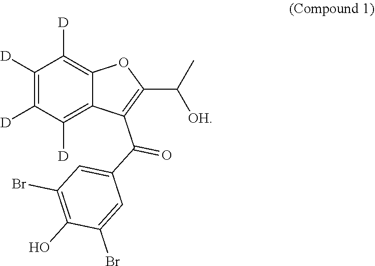 [SMILES: [2H]c1c([2H])c([2H])c2c(C(=O)c3cc(Br)c(O)c(Br)c3)c(C(C)O)oc2c1[2H]]

34. A compound having a structure selected from: 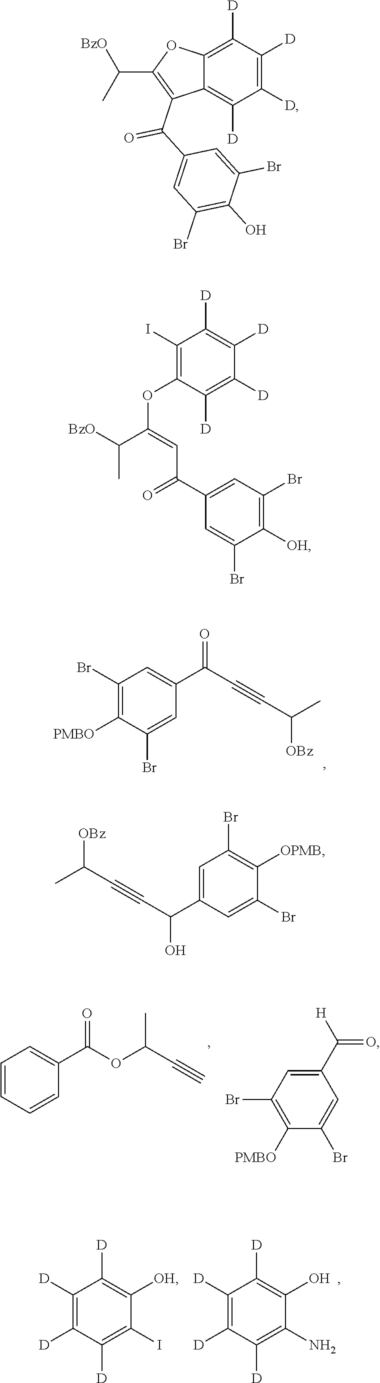 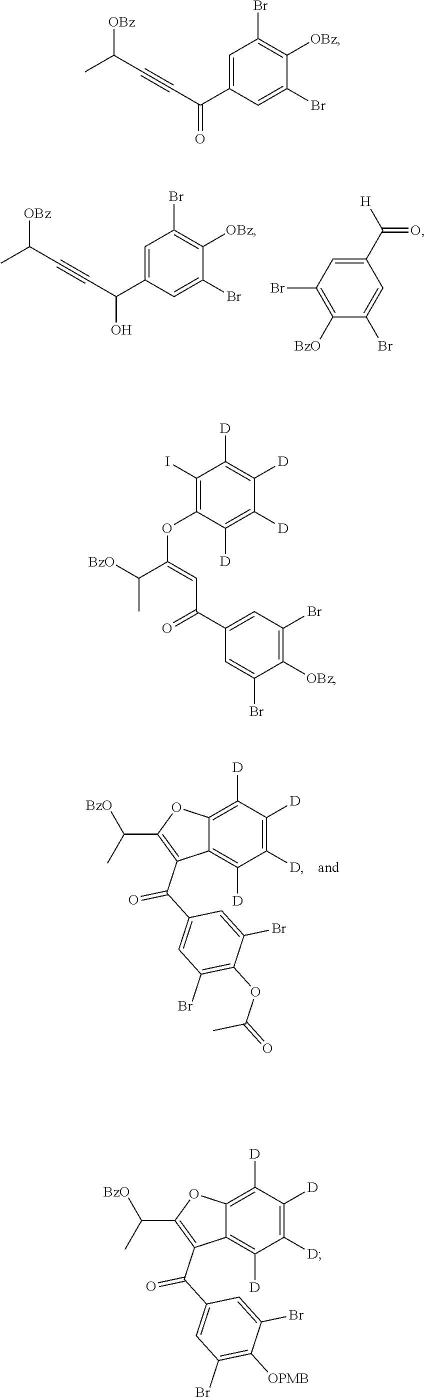
or a pharmaceutically acceptable salt thereof.
